(19) United States
(12) Patent Application Publication
(10) Pub. No.: US 2026/0234148 A1
(43) Pub. Date: Aug. 13, 2026

(54) Substituted Imidazopyrazine Compounds as IRAK3 Binders

(71) Applicant: Celgene Corporation, Princeton, NJ (US)

(72) Inventors: Farid van der Mei, Cambridge, MA (US); Guobin Miao, Lexington, MA (US); Rulin Ma, Winchester, MA (US); Laura Akullian D'Agostino, Sudbury, MA (US); Kurt Armbrust, Cambridge, MA (US)

(73) Assignee: Celgene Corporation, Princeton, NJ (US)

(21) Appl. No.: 18/997,399

(22) PCT Filed: Jul. 24, 2023
(86) PCT No.: PCT/US2023/070823

§ 371 Date: Jan. 21, 2025

Related U.S. Application Data
(60) Provisional application No. 63/391,972, filed on Jul. 25, 2022.

(51) Int. Cl. C07D 487/04; A61K 31/4985

(52) U.S. Cl. CPC C07D 487/04; A61K 31/4985

(57) ABSTRACT

Provided herein are compounds and compositions thereof that bind to IRAK3.

### CROSS-REFERENCE TO RELATED APPLICATIONS

This application claims priority to U.S. Provisional Application No. 63/391,972, filed on Jul. 25, 2022, the disclosure of which is incorporated herein by reference in its entirety for any purpose.

### FIELD

The present disclosure relates generally to compounds, compositions, and methods for their preparation and use of the compounds and compositions for binding IRAK3.

### BACKGROUND

The recruitment of immune cells to sites of injury involves the concerted interactions of a large number of soluble mediators. Several cytokines appear to play key roles in these processes, including interleukin-1 (IL-1). IL-1 produces proinflammatory responses and contributes to the tissue degeneration observed in chronic inflammatory conditions. IL-1 has also been implicated in the process of bone resorption and adipose tissue regulation. Thus, IL-1 plays a key role in a large number of pathological conditions including rheumatoid arthritis, inflammatory bowel disease, multiple sclerosis, diabetes, obesity, cancer, and sepsis.

IL-1 treatment of cells induces the formation of a complex consisting of the two IL-1 receptor chains, IL-1R1 and IL-1RAcP, and the resulting heterodimer recruits an adaptor molecule designated as MyD88, which binds to IL-1 receptor associated kinase (IRAK) (Wesche et al., J. Biol. Chem. 1999, 274, 19403-19410; O'Neill et al., J. Leukoc. Biol. 1998, 63, 650-657; Auron, Cytokine Growth Factor Rev. 1998, 9:221-237; and O'Neill, Biochem. Soc. Trans. 2000, 28, 557-563). Four members of the IRAK family have been identified: IRAK1, IRAK2, IRAK3, and IRAK4. These proteins are characterized by a typical N-terminal death domain that mediates interaction with MyD88-family adaptor proteins and a centrally located kinase domain. Of the four members in the mammalian IRAK family, IRAK2 and IRAK3 are thought to be catalytically inactive pseudokinases (Wesche et al., J. Biol. Chem. 1999, 274, 19403-19410), but the detailed roles of the two kinases are still largely unknown (Lagne et al., Structure 2021, 29, 238-251). Nonetheless, reports indicate the association of IRAK3 with negative regulation of TLR (toll-like receptor) signaling which is involved in detecting microorganisms and protecting multicellular organisms from infection (Kobayashi et al., Cell 2002, 110, 191-202). More recent studies have revealed the linkage between mutation or high expression levels of IRAK3 and various diseases such as asthma and cancer (Balaci et al., Am. J. Hum. Genet. 2007, 80 (6), 1103-1114; Kesselring et al., Cancer Cell 2016, 29 (5), 685-696), which suggest the potential of IRAK3 as a drug target and the need for IRAK3 binding small molecules.

Accordingly, in one aspect, provided herein are compounds that bind to IRAK3.

### SUMMARY

Described herein, in certain embodiments, are compounds and compositions thereof that bind to IRAK3. In various embodiments, the compounds and compositions thereof may be used for treatment of inflammatory or autoimmune disease, or cancer.

The present embodiments can be understood more fully by reference to the detailed description and examples, which are intended to exemplify non-limiting embodiments.

Embodiment A1. A compound of Formula (I):

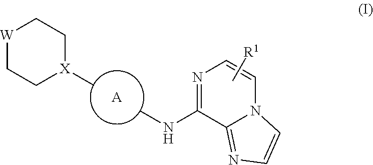

or a pharmaceutically acceptable salt thereof, wherein:
Ring A is C₆-C₁₀ aryl or 5- to 6-membered heteroaryl, wherein the heteroaryl contains 1-3 heteroatoms selected from nitrogen, oxygen, and sulfur;
R¹ is H, C₆-C₁₀ aryl, or C₃-C₆ cycloalkyl, wherein the aryl and cycloalkyl are optionally substituted by 1-5 substituents selected from C₁-C₆ alkyl, C₁-C₆ haloalkyl, C₁-C₆ alkoxy, halo, —OH, and —CN;
W is NR² or CR^{3a}R^{3b};
R² is H, C₁-C₆ alkyl, C₁-C₆ haloalkyl, or —C(O)(C₁-C₆ alkyl); R^{3a} and R^{3b} are independently H, C₁-C₆ alkyl, C₁-C₆ haloalkyl, C₁-C₆ alkoxy, or —C(O)(C₁-C₆ alkyl); and
X is CH or N.

Embodiment A2. The compound of embodiment A1, or a pharmaceutically acceptable salt thereof, wherein:
Ring A is C₆-C₁₀ aryl.

Embodiment A3. The compound of embodiment A1, or a pharmaceutically acceptable salt thereof, wherein:

Ring A is 5- to 6-membered heteroaryl, wherein the heteroaryl contains 1-3 heteroatoms selected from nitrogen, oxygen, and sulfur.

Embodiment A4. The compound of any one of embodiments A1-A3, or a pharmaceutically acceptable salt thereof, wherein:
Ring A is 7

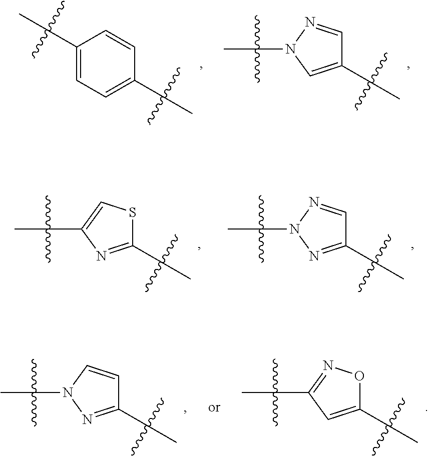 [SMILES: *c1ccc(*)cc1 ; *c1cnn(*)c1 ; *c1csc(*)n1 ; *c1cnn(*)n1 ; *c1ccn(*)n1 ; *c1cc(*)on1]

Embodiment A5. The compound of any one of embodiments A1-A4, or a pharmaceutically acceptable salt thereof, wherein R¹ is H.

Embodiment A6. The compound of any one of embodiments A1-A4, or a pharmaceutically acceptable salt thereof, wherein R¹ is C₃-C₆ cycloalkyl optionally substituted by 1-5 substituents selected from C₁-C₆ alkyl, C₁-C₆ haloalkyl, C₁-C₆ alkoxy, halo, —OH, and —CN.

Embodiment A7. The compound of embodiment A6, or a pharmaceutically acceptable salt thereof, wherein R¹ is C₃-C₆ cycloalkyl.

Embodiment A8. The compound of any one of embodiments A1-A4, or a pharmaceutically acceptable salt thereof, wherein R¹ is C₆-C₁₀ aryl optionally substituted by 1-5 substituents selected from C₁-C₆ alkyl, C₁-C₆ haloalkyl, C₁-C₆ alkoxy, halo, —OH, and —CN.

Embodiment A9. The compound of any one of embodiments A1-A4, A6, and A7, or a pharmaceutically acceptable salt thereof, wherein:
R¹ is

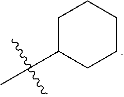 [SMILES: *C1CCCCC1]

Embodiment A10. The compound of any one of embodiments A1-A4 and A8, or a pharmaceutically acceptable salt thereof, wherein:
R¹ is

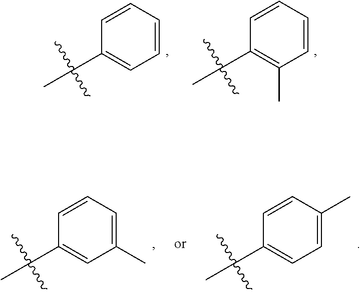

Embodiment A11. The compound of any one of embodiments A1-A10, or a pharmaceutically acceptable salt thereof, wherein:
W is NR² or CR^{3a}R^{3b},
R² is H, C₁-C₆ alkyl, or —C(O)(C₁-C₃ alkyl); and
R^{3a} and R^{3b} are independently H, C₁-C₃ alkyl, or —C(O)(C₁-C₆ alkyl).

Embodiment A12. The compound of embodiment A11, or a pharmaceutically acceptable salt thereof, wherein:
W is NR²; and
R² is H, C₁-C₆ alkyl, or —C(O)(C₁-C₃ alkyl).

Embodiment A13. The compound of any one of embodiments A1-A10, or a pharmaceutically acceptable salt thereof, wherein X is CH.

Embodiment A14. The compound of any one of embodiments A1-A10, or a pharmaceutically acceptable salt thereof, wherein X is N.

Embodiment A15. The compound of any one of embodiments A1-A13, or a pharmaceutically acceptable salt thereof, wherein:

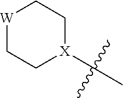

is

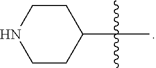 [SMILES: *C1CCNCC1]

Embodiment A16. The compound of any one of embodiments A1-A12 and A14, or a pharmaceutically acceptable salt thereof, wherein:

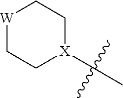

is

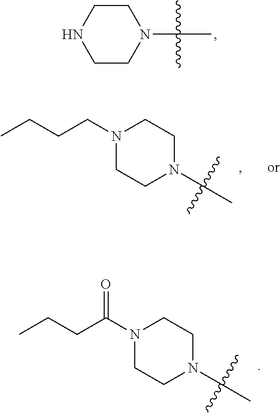 [SMILES: *N1CCNCC1 ; *N1CCN(CCCC)CC1 ; *N1CCN(C(=O)CCC)CC1]

Embodiment A17. The compound of any one of embodiments A1-A16, or a pharmaceutically acceptable salt thereof, wherein the compound is Formula (II) or (III):

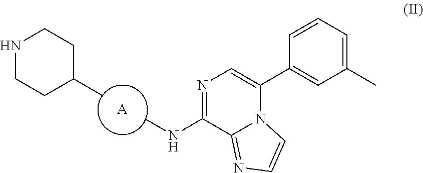

wherein Ring A is 5-membered heteroaryl that contains 1-3 heteroatoms selected from nitrogen, oxygen, sulfur;

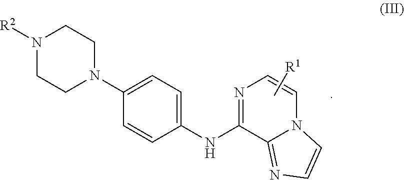

Embodiment A18. A compound selected from the compounds of Table 1 or a pharmaceutically acceptable salt thereof.

Embodiment A19. A pharmaceutical composition comprising the compound of any one of embodiments A1-A18, or a pharmaceutically acceptable salt thereof, and a pharmaceutically acceptable excipient.

Embodiment A20. A method of binding Interleukin-1 Receptor-Associated Kinase 3 (IRAK3) comprising contacting IRAK3 with an effective amount of the compound of any one of embodiments A1-A18, or a pharmaceutically acceptable salt thereof, or the pharmaceutical composition of embodiment A19.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean ±20%, ±10%, ±5%, or ±1% of the indicated range, value, or structure, unless otherwise indicated.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈alkyl) or, in some embodiments, from 1 to 6 (C₁-C₆ alkyl), 1 to 4 (C₁-C₄ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH≡CH(CH₃), —CH≡C(CH₃)₂, —C(CH₃)≡CH₂, —C(CH₃)—CH(CH₃), —C(CH₂CH₃)≡CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy; oxo (═O); amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₈ cycloalkyl), whereas in other embodiments the number of ring carbon atoms ranges from 3 to 5 (C₃-C₅ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

A “heterocyclyl” is a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2 (1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

An “aryl” group is an aromatic carbocyclic group of from 6 to 14 carbon atoms (C₆-C₁₄ aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1 (2H)-onyl), tetrahydroquinolinyl, quinoxalinyl, and quinazolinyl groups. A heteroaryl group can be substituted or unsubstituted.

A “halogen” or “halo” is fluorine, chlorine, bromine or iodine.

An “alkoxy” group is —O-(alkyl), wherein alkyl is defined above.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl group has one to six carbon atoms and is substituted by one or more halo radicals (C₁-C₆ haloalkyl), or the haloalkyl group has one to three carbon atoms and is substituted by one or more halo radicals (C₁-C₃ haloalkyl). The halo radicals may be all the same or the halo radicals may be different. Unless specifically stated otherwise, a haloalkyl group is optionally substituted.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Embodiments of the disclosure are meant to encompass pharmaceutically acceptable salts, tautomers, isotopologues, and stereoisomers of the compounds provided herein, such as the compounds of Formula (I).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of Formula (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuja, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

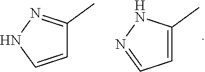

As readily understood by one skilled in the art, a wide variety of functional groups and other structures may exhibit tautomerism and all tautomers of compounds of Formula (I) are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a neurodegenerative disease, as described herein, or a symptom thereof.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is a neurodegenerative disease, as described herein, or symptoms thereof.

The term “effective amount” in connection with a compound disclosed herein means an amount capable of treating or preventing a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an IRAK3 mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein is a compound of Formula (I):

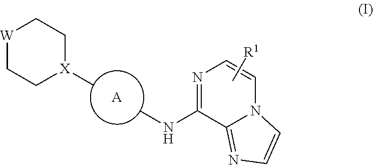

or a pharmaceutically acceptable salt thereof, wherein:
   Ring A is C₆-C₁₀ aryl or 5- to 6-membered heteroaryl, wherein the heteroaryl contains 1-3 heteroatoms selected from nitrogen, oxygen, and sulfur;
   R¹ is H, C₆-C₁₀ aryl, or C₃-C₆ cycloalkyl, wherein the aryl and cycloalkyl are optionally substituted by 1-5 substituents selected from C₁-C₆ alkyl, C₁-C₆ haloalkyl, C₁-C₆ alkoxy, halo, —OH, and —CN;
   W is NR² or CR^{3a}R^{3b};
   R² is H, C₁-C₆ alkyl, C₁-C₆ haloalkyl, C₁-C₆ alkoxy, or —C(O)(C₁-C₆ alkyl);
   R^{3a} and R^{3b} are independently H, C₁-C₆ alkyl, C₁-C₆ haloalkyl, C₁-C₆ alkoxy, or —C(O)(C₁-C₆ alkyl); and
   X is CH or N.

In some embodiments, Ring A is C₆-C₁₀ aryl or 5- to 6-membered heteroaryl, wherein the heteroaryl contains 1-3 heteroatoms selected from nitrogen, oxygen, and sulfur. In some embodiments, Ring A is C₆-C₁₀ aryl or 5- to 6-membered heteroaryl, wherein the heteroaryl contains 2-3 heteroatoms selected from nitrogen, oxygen, and sulfur. In some embodiments, Ring A is phenyl or 5-membered heteroaryl, wherein the heteroaryl contains 2-3 heteroatoms selected from nitrogen, oxygen, and sulfur. In some embodiments, Ring A is phenyl. In some embodiments, Ring A is 5-membered heteroaryl containing 2-3 heteroatoms selected from nitrogen, oxygen, and sulfur.

In some embodiments, Ring A is C₆-C₁₀ aryl. In some embodiments, Ring A is C₆ aryl. In some embodiments, Ring A is phenyl. In some embodiments, Ring A is C₁₀ aryl. In some embodiments, Ring A is naphthyl. In some embodiments, Ring A is

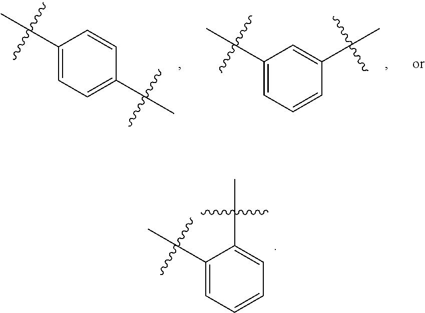 [SMILES: *c1ccc(*)cc1 ; *c1cccc(*)c1 ; *c1ccccc1*]

In some embodiments, Ring A is

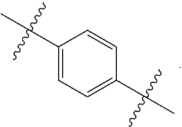 [SMILES: *c1ccc(*)cc1]

In some embodiments, Ring A is 5- to 6-membered heteroaryl containing 1-3 heteroatoms selected from nitrogen, oxygen, and sulfur. In some embodiments, Ring A is 5- to 6-membered heteroaryl containing one heteroatom selected from nitrogen, oxygen, and sulfur. In some embodiments, Ring A is 5- to 6-membered heteroaryl containing 2-3 heteroatoms selected from nitrogen, oxygen, and sulfur. In some embodiments, Ring A is 5- to 6-membered heteroaryl containing 2 heteroatoms selected from nitrogen, oxygen, and sulfur. In some embodiments, Ring A is 5- to 6-membered heteroaryl containing 3 heteroatoms selected from nitrogen, oxygen, and sulfur. In some embodiments, Ring A is 5-membered heteroaryl containing one heteroatom selected from nitrogen, oxygen, and sulfur. In some embodiments, Ring A is 5-membered heteroaryl containing 2-3 heteroatoms selected from nitrogen, oxygen, and sulfur. In some embodiments, Ring A is 5-membered heteroaryl containing 2 nitrogen atoms. In some embodiments, Ring A is 5-membered heteroaryl containing 3 nitrogen atoms.

In some embodiments, Ring A is 5-membered heteroaryl containing one nitrogen atom and one sulfur atom. In some embodiments, Ring A is 5-membered heteroaryl containing one nitrogen atom and one oxygen atom. In some embodiments, Ring A is pyrrolyl, pyrazolyl, triazolyl, imidazolyl, thiazolyl, oxazolyl, isoxazolyl, furanyl, or thiadiazolyl. In some embodiments, Ring A is 6-membered heteroaryl containing one heteroatom selected from nitrogen, oxygen, and sulfur. In some embodiments, Ring A is 6-membered heteroaryl containing 2-3 heteroatoms selected from nitrogen, oxygen, and sulfur. In some embodiments, Ring A is 6-membered heteroaryl containing 2 heteroatoms selected from nitrogen, oxygen, and sulfur. In some embodiments, Ring A is 6-membered heteroaryl containing 3 heteroatoms selected from nitrogen, oxygen, and sulfur. In some embodiments, Ring A is 6-membered heteroaryl containing 2 nitrogen atoms. In some embodiments, Ring A is 6-membered heteroaryl containing 3 nitrogen atoms. In some embodiments, Ring A is 6-membered heteroaryl containing one nitrogen atom and one sulfur atom. In some embodiments, Ring A is 6-membered heteroaryl containing one nitrogen atom and one oxygen atom. In some embodiments, Ring A is pyridinyl, pyrazinyl, pyrimidinyl, pyridazinyl, or triazinyl. In some embodiments, Ring A is

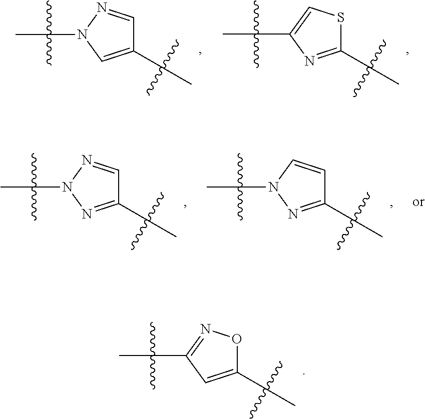 [SMILES: *c1cnn(*)c1 ; *c1csc(*)n1 ; *c1cnn(*)n1 ; *c1ccn(*)n1 ; *c1cc(*)on1]

In some embodiments, R¹ is H, C₆-C₁₀ aryl, or C₃-C₆ cycloalkyl, wherein the aryl and cycloalkyl are optionally substituted by 1-5 substituents selected from C₁-C₆ alkyl, C₁-C₆ haloalkyl, C₁-C₆ alkoxy, halo, —OH, and —CN. In some embodiments, R¹ is H, C₆-C₁₀ aryl, or C₃-C₆ cycloalkyl, wherein the aryl and cycloalkyl are optionally substituted by 5 substituents selected from C₁-C₆ alkyl, C₁-C₆ haloalkyl, C₁-C₆ alkoxy, halo, —OH, and —CN. In some embodiments, R¹ is H, C₆-C₁₀ aryl, or C₃-C₆ cycloalkyl, wherein the aryl and cycloalkyl are optionally substituted by 4 substituents selected from C₁-C₆ alkyl, C₁-C₆ haloalkyl, C₁-C₆ alkoxy, halo, —OH, and —CN. In some embodiments, R¹ is H, C₆-C₁₀ aryl, or C₃-C₆ cycloalkyl, wherein the aryl and cycloalkyl are optionally substituted by 1-3 substituents selected from C₁-C₆ alkyl, C₁-C₆ haloalkyl, C₁-C₆ alkoxy, halo, —OH, and —CN. In some embodiments, R¹ is H, C₆-C₁₀ aryl, or C₃-C₆ cycloalkyl, wherein the aryl and cycloalkyl are optionally substituted by 3 substituents selected from C₁-C₆ alkyl, C₁-C₆ haloalkyl, C₁-C₆ alkoxy, halo, —OH, and —CN. In some embodiments, R¹ is H, C₆-C₁₀ aryl, or C₃-C₆ cycloalkyl, wherein the aryl and cycloalkyl are optionally substituted by 2 substituents selected from C₁-C₆ alkyl, C₁-C₆ haloalkyl, C₁-C₆ alkoxy, halo, —OH, and —CN. In some embodiments, R¹ is H, C₆-C₁₀ aryl, or C₃-C₆ cycloalkyl, wherein the aryl and cycloalkyl are optionally substituted by 1 substituent selected from C₁-C₆ alkyl, C₁-C₆ haloalkyl, C₁-C₆ alkoxy, halo, —OH, and —CN. In some embodiments, R¹ is H, C₆-C₁₀ aryl, or C₃-C₆ cycloalkyl, wherein the aryl and cycloalkyl are optionally substituted by 1-3 substituents selected from C₁-C₃ alkyl, C₁-C₃ haloalkyl, C₁-C₃ alkoxy, halo, —OH, and —CN. In some embodiments, R¹ is H, C₆-C₁₀ aryl, or C₃-C₆ cycloalkyl, wherein the aryl and cycloalkyl are optionally substituted by 3 substituents selected from C₁-C₃ alkyl, C₁-C₃ haloalkyl, C₁-C₃ alkoxy, halo, —OH, and —CN. In some embodiments, R¹ is H, C₆-C₁₀ aryl, or C₃-C₆ cycloalkyl, wherein the aryl and cycloalkyl are optionally substituted by 2 substituents selected from C₁-C₃ alkyl, C₁-C₃ haloalkyl, C₁-C₃ alkoxy, halo, —OH, and —CN. In some embodiments, R¹ is H, C₆-C₁₀ aryl, or C₃-C₆ cycloalkyl, wherein the aryl and cycloalkyl are optionally substituted by 1 substituent selected from C₁-C₃ alkyl, C₁-C₃ haloalkyl, C₁-C₃ alkoxy, halo, —OH, and —CN. In some embodiments, R¹ is H, phenyl, or cyclohexyl, wherein the phenyl and cyclohexyl are optionally substituted by 1-3 substituents selected from C₁-C₃ alkyl, C₁-C₃ haloalkyl, C₁-C₃ alkoxy, halo, —OH, and —CN. In some embodiments, R¹ is H, phenyl, or cyclohexyl, wherein the phenyl and cyclohexyl are optionally substituted by 3 substituents selected from C₁-C₃ alkyl, C₁-C₃ haloalkyl, C₁-C₃ alkoxy, halo, —OH, and —CN. In some embodiments, R¹ is H, phenyl, or cyclohexyl, wherein the phenyl and cyclohexyl are optionally substituted by 2 substituents selected from C₁-C₃ alkyl, C₁-C₃ haloalkyl, C₁-C₃ alkoxy, halo, —OH, and —CN. In some embodiments, R¹ is H, phenyl, or cyclohexyl, wherein the phenyl and cyclohexyl are optionally substituted by 1 substituent selected from C₁-C₃ alkyl, C₁-C₃ haloalkyl, C₁-C₃ alkoxy, halo, —OH, and —CN. In some embodiments, R¹ is H, phenyl, or cyclohexyl, wherein the phenyl and cyclohexyl are optionally substituted by 1 substituent selected from propyl, ethyl, methyl, —CH₂CH₂CF₃, —CH₂CH₂CHF₂, —CH₂CH₂CH₂F, —CH₂CF₃, —CH₂CHF₂, —CH₂CH₂F, —CF₃, —CHF₂, —CH₂F, —OCH₂CH₂CH₃, —OCH₂CH₃, —OCH₃, halo, —OH, and —CN. In some embodiments, R¹ is H, phenyl, or cyclohexyl, wherein the phenyl and cyclohexyl are optionally substituted by 1 substituent selected from methyl, —CF₃, —CHF₂, —CH₂F, —OCH₃, halo, —OH, and —CN. In some embodiments, R¹ is H, phenyl, or cyclohexyl, wherein the phenyl and cyclohexyl are optionally substituted by methyl. In some embodiments, R¹ is H. In some embodiments, R¹ is phenyl optionally substituted by methyl. In some embodiments, R¹ is cyclohexyl optionally substituted by methyl. In some embodiments, R¹ is cyclohexyl.

In some embodiments, R¹ is H.

In some embodiments, R¹ is C₆-C₁₀ aryl optionally substituted by 1-5 substituents selected from C₁-C₆ alkyl, C₁-C₆ haloalkyl, C₁-C₆ alkoxy, halo, —OH, and —CN. In some embodiments, R¹ is C₆-C₁₀ aryl optionally substituted by 5 substituents selected from C₁-C₆ alkyl, C₁-C₆ haloalkyl, C₁-C₆ alkoxy, halo, —OH, and —CN. In some embodiments, R¹ is C₆-C₁₀ aryl optionally substituted by 4 substituents selected from C₁-C₆ alkyl, C₁-C₆ haloalkyl, C₁-C₆ alkoxy, halo, —OH, and —CN. In some embodiments, R¹ is C₆-C₁₀ aryl optionally substituted by 1-3 substituents selected from C₁-C₆ alkyl, C₁-C₆ haloalkyl, C₁-C₆ alkoxy, halo, —OH, and —CN. In some embodiments, R¹ is C₆-C₁₀ aryl optionally substituted by 3 substituents selected from C₁-C₆ alkyl, C₁-C₆ haloalkyl, C₁-C₆ alkoxy, halo, —OH, and —CN. In some embodiments, R¹ is C₆-C₁₀ aryl optionally substituted by 2 substituents selected from C₁-C₆ alkyl, C₁-C₆ haloalkyl, C₁-C₆ alkoxy, halo, —OH, and —CN. In some embodiments, R¹ is C₆-C₁₀ aryl optionally substituted by 1 substituent selected from C₁-C₆ alkyl, C₁-C₆ haloalkyl, C₁-C₆ alkoxy, halo, —OH, and —CN. In some embodiments, R¹ is C₆-C₁₀ aryl optionally substituted by 1-3 substituents selected from C₁-C₃ alkyl, C₁-C₃ haloalkyl, C₁-C₃ alkoxy, halo, —OH, and —CN. In some embodiments, R¹ is C₆-C₁₀ aryl optionally substituted by 3 substituents selected from C₁-C₃ alkyl, C₁-C₃ haloalkyl, C₁-C₃ alkoxy, halo, —OH, and —CN. In some embodiments, R¹ is C₆-C₁₀ aryl optionally substituted by 2 substituents selected from C₁-C₃ alkyl, C₁-C₃ haloalkyl, C₁-C₃ alkoxy, halo, —OH, and —CN. In some embodiments, R¹ is C₆-C₁₀ aryl optionally substituted by 1 substituent selected from C₁-C₃ alkyl, C₁-C₃ haloalkyl, C₁-C₃ alkoxy, halo, —OH, and —CN. In some embodiments, R¹ is phenyl optionally substituted by 1-3 substituents selected from C₁-C₃ alkyl, C₁-C₃ haloalkyl, C₁-C₃ alkoxy, halo, —OH, and —CN. In some embodiments, R¹ is phenyl optionally substituted by 2 substituents selected from C₁-C₃ alkyl, C₁-C₃ haloalkyl, C₁-C₃ alkoxy, halo, —OH, and —CN. In some embodiments, R¹ is phenyl optionally substituted by 1 substituent selected from C₁-C₃ alkyl, C₁-C₃ haloalkyl, C₁-C₃ alkoxy, halo, —OH, and —CN. In some embodiments, R¹ is phenyl optionally substituted by 1 substituent selected from propyl, ethyl, methyl, —CH₂CH₂CF₃, —CH₂CH₂CHF₂, —CH₂CH₂CH₂F, —CH₂CF₃, —CH₂CHF₂, —CH₂CH₂F, —CF₃, —CHF₂, —CH₂F, —OCH₂CH₂CH₃, —OCH₂CH₃, —OCH₃, halo, —OH, and —CN. In some embodiments, R¹ is phenyl optionally substituted by 1 substituent selected from methyl, —CF₃, —CHF₂, —CH₂F, —OCH₃, halo, —OH, and —CN. In some embodiments, R¹ is phenyl optionally substituted by methyl. In some embodiments, R¹ is

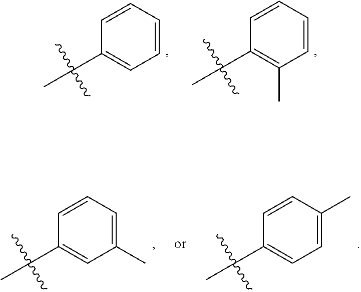

In some embodiments, R¹ is C₃-C₆ cycloalkyl optionally substituted by 1-5 substituents selected from C₁-C₆ alkyl, C₁-C₆ haloalkyl, C₁-C₆ alkoxy, halo, —OH, and —CN. In some embodiments, R¹ is cyclopropyl optionally substituted by 1-5 substituents selected from C₁-C₆ alkyl, C₁-C₆ haloalkyl, C₁-C₆ alkoxy, halo, —OH, and —CN. In some embodiments, R¹ is cyclobutyl optionally substituted by 1-5 substituents selected from C₁-C₆ alkyl, C₁-C₆ haloalkyl, C₁-C₆ alkoxy, halo, —OH, and —CN. In some embodiments, R¹ is cyclopentyl optionally substituted by 1-5 substituents selected from C₁-C₆ alkyl, C₁-C₆ haloalkyl, C₁-C₆ alkoxy, halo, —OH, and —CN. In some embodiments, R¹ is cyclohexyl optionally substituted by 1-5 substituents selected from C₁-C₆ alkyl, C₁-C₆ haloalkyl, C₁-C₆ alkoxy, halo, —OH, and —CN. In some embodiments, R¹ is C₃-C₆ cycloalkyl optionally substituted by 5 substituents selected from C₁-C₆ alkyl, C₁-C₆ haloalkyl, C₁-C₆ alkoxy, halo, —OH, and —CN. In some embodiments, R¹ is C₃-C₆ cycloalkyl optionally substituted by 4 substituents selected from C₁-C₆ alkyl, C₁-C₆ haloalkyl, C₁-C₆ alkoxy, halo, —OH, and —CN. In some embodiments, R¹ is C₃-C₆ cycloalkyl optionally substituted by 1-3 substituents selected from C₁-C₆ alkyl, C₁-C₆ haloalkyl, C₁-C₆ alkoxy, halo, —OH, and —CN. In some embodiments, R¹ is C₃-C₆ cycloalkyl optionally substituted by 3 substituents selected from C₁-C₆ alkyl, C₁-C₆ haloalkyl, C₁-C₆ alkoxy, halo, —OH, and —CN. In some embodiments, R¹ is C₃-C₆ cycloalkyl optionally substituted by 2 substituents selected from C₁-C₆ alkyl, C₁-C₆ haloalkyl, C₁-C₆ alkoxy, halo, —OH, and —CN. In some embodiments, R¹ is C₃-C₆ cycloalkyl optionally substituted by 1 substituent selected from C₁-C₆ alkyl, C₁-C₆ haloalkyl, C₁-C₆ alkoxy, halo, —OH, and —CN. In some embodiments, R¹ is C₃-C₆ cycloalkyl optionally substituted by 1-3 substituents selected from C₁-C₃ alkyl, C₁-C₃ haloalkyl, C₁-C₃ alkoxy, halo, —OH, and —CN. In some embodiments, R¹ is C₃-C₆ cycloalkyl optionally substituted by 3 substituents selected from C₁-C₃ alkyl, C₁-C₃ haloalkyl, C₁-C₃ alkoxy, halo, —OH, and —CN. In some embodiments, R¹ is C₃-C₆ cycloalkyl optionally substituted by 2 substituents selected from C₁-C₃ alkyl, C₁-C₃ haloalkyl, C₁-C₃ alkoxy, halo, —OH, and —CN. In some embodiments, R¹ is C₃-C₆ cycloalkyl optionally substituted by 1 substituent selected from C₁-C₃ alkyl, C₁-C₃ haloalkyl, C₁-C₃ alkoxy, halo, —OH, and —CN. In some embodiments, R¹ is cyclohexyl optionally substituted by 1-3 substituents selected from C₁-C₃ alkyl, C₁-C₃ haloalkyl, C₁-C₃ alkoxy, halo, —OH, and —CN. In some embodiments, R¹ is cyclohexyl optionally substituted by 3 substituents selected from C₁-C₃ alkyl, C₁-C₃ haloalkyl, C₁-C₃ alkoxy, halo, —OH, and —CN. In some embodiments, R¹ is cyclohexyl optionally substituted by 2 substituents selected from C₁-C₃ alkyl, C₁-C₃ haloalkyl, C₁-C₃ alkoxy, halo, —OH, and —CN. In some embodiments, R¹ is cyclohexyl optionally substituted by 1 substituent selected from C₁-C₃ alkyl, C₁-C₃ haloalkyl, C₁-C₃ alkoxy, halo, —OH, and —CN. In some embodiments, R¹ is cyclohexyl optionally substituted by 1 substituent selected from propyl, ethyl, methyl, —CH₂CH₂CF₃, —CH₂CH₂CHF₂, —CH₂CH₂CH₂F, —CH₂CF₃, —CH₂CHF₂, —CH₂CH₂F, —CF₃, —CHF₂, —CH₂F, —OCH₂CH₂CH₃, —OCH₂CH₃, —OCH₃, halo, —OH, and —CN. In some embodiments, R¹ is cyclohexyl optionally substituted by 1 substituent selected from methyl, —CF₃, —CHF₂, —CH₂F, —OCH₃, halo, —OH, and —CN. In some embodiments, R¹ is cyclohexyl optionally substituted by methyl. In some embodiments, R¹ is unsubstituted cyclohexyl. In some embodiments, R¹ is

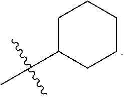 [SMILES: *C1CCCCC1]

In some embodiments, W is NR² or CR^{3a}R^{3b}. In some embodiments, W is NR². In some embodiments, W is CR^{3a}R^{3b}.

In some embodiments, R² is H, C₁-C₆ alkyl, C₁-C₆ haloalkyl, C₁-C₆ alkoxy, or —C(O)(C₁-C₆ alkyl). In some embodiments, R² is H, C₁-C₄ alkyl, C₁-C₃ haloalkyl, C₁-C₃ alkoxy, or —C(O)(C₁-C₃ alkyl). In some embodiments, R² is H, methyl, ethyl, propyl, butyl, —CH₂CH₂CF₃, —CH₂CH₂CHF₂, —CH₂CH₂CH₂F, —CH₂CF₃, —CH₂CHF₂, —CH₂CH₂F, —CF₃, —CHF₂, —CH₂F, —OCH₂CH₂CH₃, —OCH₂CH₃, —OCH₃, —C(O)(CH₃), —C(O)(CH₂CH₃), or —C(O)(CH₂CH₂CH₃). In some embodiments, R² is H, butyl, or —C(O)(CH₂CH₂CH₃). In some embodiments, R² is H. In some embodiments, R² is butyl. In some embodiments, R² is —C(O)(CH₂CH₂CH₃).

In some embodiments, R² is H.

In some embodiments, R² is C₁-C₆ alkyl. In some embodiments, R² is C₁-C₄ alkyl. In some embodiments, R² is methyl, ethyl, propyl, or butyl. In some embodiments, R² is butyl.

In some embodiments, R² is C₁-C₆ haloalkyl. In some embodiments, R² is C₁-C₃ haloalkyl. In some embodiments, R² is —CH₂CH₂CF₃, —CH₂CH₂CHF₂, —CH₂CH₂CH₂F, —CH₂CF₃, —CH₂CHF₂, —CH₂CH₂F, —CF₃, —CHF₂, or —CH₂F. In some embodiments, R² is —CF₃, —CHF₂, or —CH₂F.

In some embodiments, R² is C₁-C₆ alkoxy. In some embodiments, R² is C₁-C₃ alkoxy. In some embodiments, R² is —OCH₂CH₂CH₃, —OCH₂CH₃, or —OCH₃. In some embodiments, R² is —OCH₃.

In some embodiments, R² is C(O)(C₁-C₆ alkyl). In some embodiments, R² is —C(O)(C₁-C₃ alkyl). In some embodiments, R² is —C(O)(CH₃), —C(O)(CH₂CH₃), or —C(O)(CH₂CH₂CH₃). In some embodiments, R² is —C(O)(CH₂CH₂CH₃).

In some embodiments, R3ª is H, C₁-C₆ alkyl, C₁-C₆ haloalkyl, C₁-C₆ alkoxy, or —C(O)(C₁-C₆ alkyl). In some embodiments, R3ª is H, C₁-C₄ alkyl, C₁-C₄ haloalkyl, C₁-C₄ alkoxy, or —C(O)(C₁-C₄ alkyl). In some embodiments, R^{3a} is H. In some embodiments, R^{3a} is butyl, propyl, ethyl, or methyl. In some embodiments, R^{3a} is —CH₂CH₂CH₂CF₃, —CH₂CH₂CH₂CHF₂, —CH₂CH₂CH₂CH₂F, —CH₂CH₂CF₃, —CH₂CH₂CHF₂, —CH₂CH₂CH₂F, —CH₂CF₃, —CH₂CHF₂, —CH₂CH₂F, —CF₃, —CHF₂, or —CH₂F. In some embodiments, R^{3a} is —OCH₂CH₂CH₂CH₃, —OCH₂CH₂CH₃, —OCH₂CH₃, or —OCH₃. In some embodiments, R^{3a} is —C(O)(CH₃), —C(O)(CH₂CH₃), —C(O)(CH₂CH₂CH₃), or —C(O)(CH₂CH₂CH₂CH₃). In some embodiments, R^{3a} is H, butyl, —CF₃, —CHF₂, —CH₂F, OCH₃, or —C(O)(CH₂CH₂CH₃).

In some embodiments, R^{3b} is H, C₁-C₆ alkyl, C₁-C₆ haloalkyl, C₁-C₆ alkoxy, or —C(O)(C₁-C₆ alkyl). In some embodiments, R^{3b} is H, C₁-C₄ alkyl, C₁-C₄ haloalkyl, C₁-C₄ alkoxy, or —C(O)(C₁-C₄ alkyl). In some embodiments, R^{3b} is H. In some embodiments, R^{3b} is butyl, propyl, ethyl, or methyl. In some embodiments, R^{3b} is —CH₂CH₂CH₂CF₃, —CH₂CH₂CH₂CHF₂, —CH₂CH₂CH₂CH₂F, —CH₂CH₂CF₃, —CH₂CH₂CHF₂, —CH₂CH₂CH₂F, —CH₂CF₃, —CH₂CHF₂, —CH₂CH₂F, —CF₃, —CHF₂, or —CH₂F. In some embodiments, R^{3b} is —OCH₂CH₂CH₂CH₃, —OCH₂CH₂CH₃, —OCH₂CH₃, or —OCH₃. In some embodiments, R^{3b} is —C(O)(CH₃), —C(O)(CH₂CH₃), —C(O)(CH₂CH₂CH₃), or —C(O)(CH₂CH₂CH₂CH₃). In some embodiments, R^{3b} is H, butyl, —CF₃, —CHF₂, —CH₂F, OCH₃, or —C(O)(CH₂CH₂CH₃).

In some embodiments, R^{3a} and R^{3b} are both H. In some embodiments, R^{3a} is C₁-C₄ alkyl and R^{3b} is H. In some embodiments, R^{3a} is butyl, propyl, ethyl, or methyl, and R^{3b} is H. In some embodiments, R^{3a} is butyl and R^{3b} is H.

In some embodiments, X is CH or N. In some embodiments, X is CH. In some embodiments, X is N.

In some embodiments,

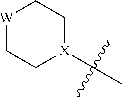

is

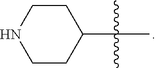 [SMILES: *C1CCNCC1]

In some embodiments,

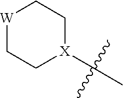

is:

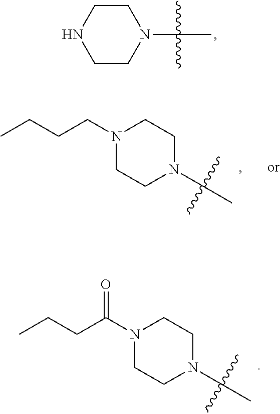 [SMILES: *N1CCNCC1 ; *N1CCN(CCCC)CC1 ; *N1CCN(C(=O)CCC)CC1]

In some embodiments, the

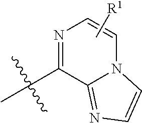

moiety of Formula (I) is

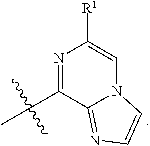

In some embodiments, the

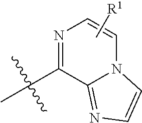

moiety of Formula (I) is

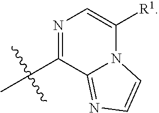

In some embodiments, the compound of Formula (I) is a compound of Formula (I-A):

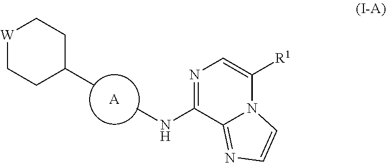

wherein Ring A, W, and R¹ are as described for Formula (I). In some variations, Ring A is 5-membered heteroaryl that contains 1-3 heteroatoms selected from nitrogen, oxygen, and sulfur.

In some embodiments, the compound of Formula (I) is a compound of Formula (I-B) or Formula (I-C):

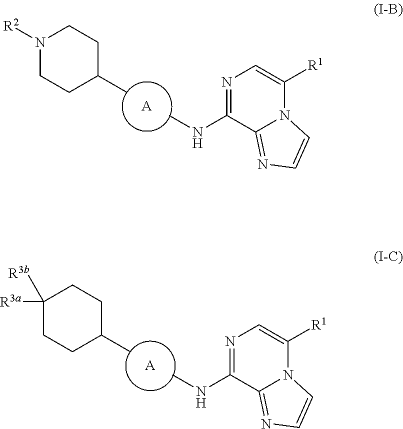

wherein Ring A, R¹, R², R^{3a}, and R^{3b} are as described for Formula (I). In some variations, Ring A is 5-membered heteroaryl that contains 1-3 heteroatoms selected from nitrogen, oxygen, and sulfur.

In some embodiments, the compound of Formula (I) is a compound of Formula (II):

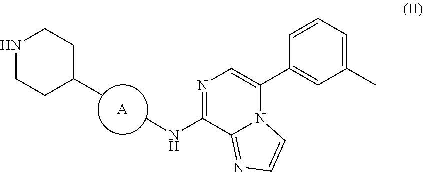

wherein Ring A is 5-membered heteroaryl that contains 1-3 heteroatoms selected from nitrogen, oxygen, and sulfur.

In some embodiments, the compound of Formula (I) is a compound of Formula (III):

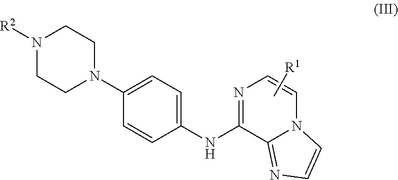

wherein R¹ and R² are as described for Formula (I).

In some embodiments, the compound of Formula (I) is a compound of Formula (III-A) or Formula (III-B):

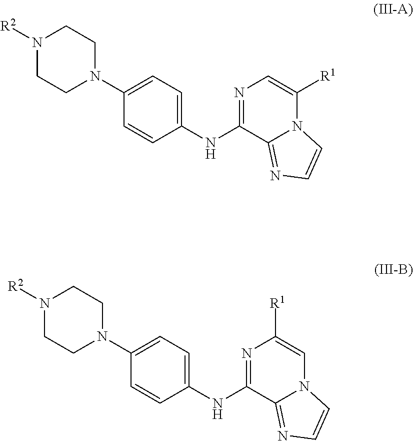

wherein R¹ and R² are as described for Formula (I).

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to R¹ of Formula (I) may be combined with every description, variation, embodiment, or aspect of Ring A, W, R², R^{3a}, R^{3b}, and X the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (I-A), (I-B), (I-C), (II), (III), (III-A), and (III-B), and are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Compound | | |
|---|---|---|
| No. | Structure | Name |
| 1 | 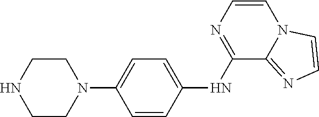 [SMILES: c1cn2ccnc2c(Nc2ccc(N3CCNCC3)cc2)n1] | N-(4-(Piperazin-1- yl)phenyl)imidazo[1,2-a] pyrazin-8-amine |
| 2 | 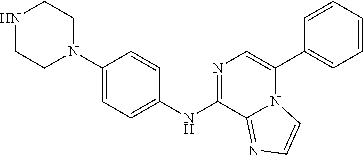 [SMILES: c1ccc(-c2cnc(Nc3ccc(N4CCNCC4)cc3)c3nccn23)cc1] | 5-Phenyl-N-(4- (piperazin-1- yl)phenyl)imidazo[1,2-a] pyrazin-8-amine |
| 3 | 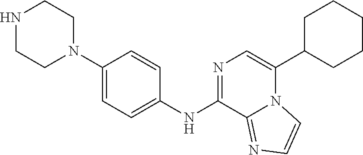 [SMILES: c1cn2c(C3CCCCC3)cnc(Nc3ccc(N4CCNCC4)cc3)c2n1] | 5-Cyclohexyl-N- (4-(piperazin-1- yl)phenyl)imidazo [1,2-a]pyrazin-8- amine |
| 4 | 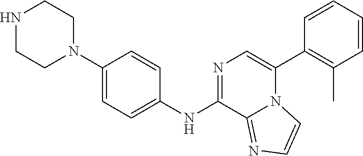 [SMILES: Cc1ccccc1-c1cnc(Nc2ccc(N3CCNCC3)cc2)c2nccn12] | N-(4-(Piperazin- 1-yl)phenyl)-5-(o- tolyl)imidazo[1,2-a] pyrazin-8-amine |
| 5 | 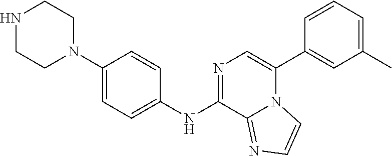 [SMILES: Cc1cccc(-c2cnc(Nc3ccc(N4CCNCC4)cc3)c3nccn23)c1] | N-(4-(Piperazin-1- yl)phenyl)-5-(m- tolyl)imidazo [1,2-a]pyrazin-8-amine |
| 6 | 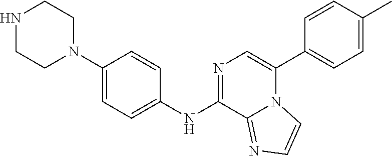 [SMILES: Cc1ccc(-c2cnc(Nc3ccc(N4CCNCC4)cc3)c3nccn23)cc1] | N-(4-(Piperazin- 1-yl)phenyl)-6-(p- tolyl)imidazo[1,2-a] pyrazin-8-amine |
| 7 | 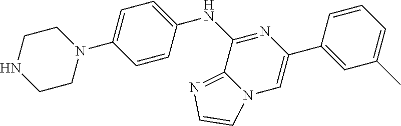 [SMILES: Cc1cccc(-c2cn3ccnc3c(Nc3ccc(N4CCNCC4)cc3)n2)c1] | N-(4-(Piperazin-1- yl)phenyl)-6-(m- tolyl)imidazo[1,2-a] pyrazin-8-amine |
| 8 | 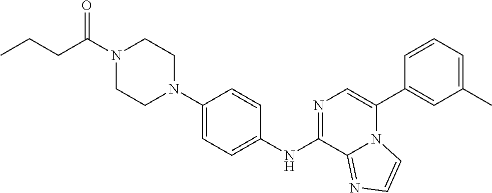 [SMILES: CCCC(=O)N1CCN(c2ccc(Nc3ncc(-c4cccc(C)c4)n4ccnc34)cc2)CC1] | 1-(4-(4-((5-(m-Tolyl) imidazo[1,2-a]pyrazin-8- yl)amino)phenyl)piperazin-1- yl)butan-1-one |
| 9 | 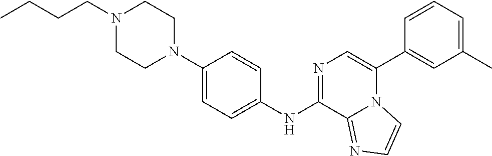 [SMILES: CCCCN1CCN(c2ccc(Nc3ncc(-c4cccc(C)c4)n4ccnc34)cc2)CC1] | N-(4-(4-Butylpiperazin- 1-yl)phenyl)-5-(m- tolyl)imidazo[1,2-a]pyrazin- 8-amine |
| 10 | 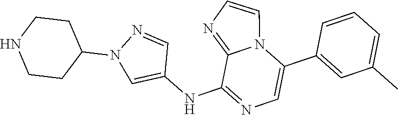 [SMILES: Cc1cccc(-c2cnc(Nc3cnn(C4CCNCC4)c3)c3nccn23)c1] | N-(1-(Piperidin-4-yl)- 1H-pyrazol-4-yl)-5-(m- tolyl)imidazo[1,2-a]pyrazin- 8-amine |
| 11 | 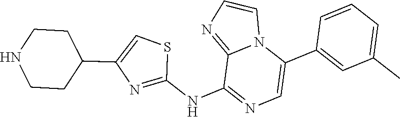 [SMILES: Cc1cccc(-c2cnc(Nc3nc(C4CCNCC4)cs3)c3nccn23)c1] | 4-(Piperidin-4-yl)-N-(5-(m- tolyl)imidazo[1,2-a]pyrazin- 8-yl)thiazol-2-amine |
| 12 | 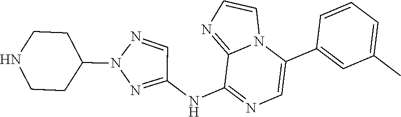 [SMILES: Cc1cccc(-c2cnc(Nc3cnn(C4CCNCC4)n3)c3nccn23)c1] | N-(2-(Piperidin- 4-yl)-2H-1,2,3- triazol-4-yl)-5-(m- tolyl)imidazo[1,2-a]pyrazin- 8-amine |
| 13 | 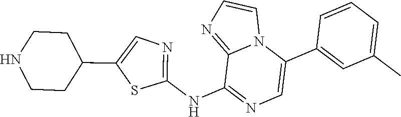 [SMILES: Cc1cccc(-c2cnc(Nc3ncc(C4CCNCC4)s3)c3nccn23)c1] | 5-(Piperidin-4-yl)-N-(5-(m- tolyl)imidazo[1,2-a]pyrazin-8- yl)thiazol-2-amine |
| 14 | 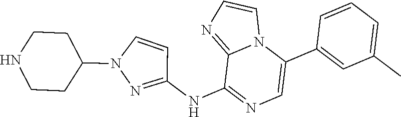 [SMILES: Cc1cccc(-c2cnc(Nc3ccn(C4CCNCC4)n3)c3nccn23)c1] | N-(1-(piperidin-4-yl)- 1H-pyrazol-3-yl)-5-(m- tolyl)imidazo[1,2-a]pyrazin- 8-amine |
| 15 | 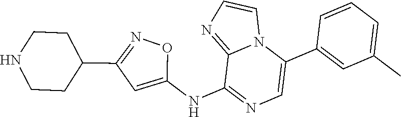 [SMILES: Cc1cccc(-c2cnc(Nc3cc(C4CCNCC4)no3)c3nccn23)c1] | 3-(Piperidin-4-yl)-N-(5-(m- tolyl)imidazo[1,2-a]pyrazin-8- yl)isoxazol-5-amine |

or a pharmaceutically acceptable salt thereof.

It is understood that in the present description, combinations of substituents and/or variables of the depicted formulae are permissible only if such contributions result in stable compounds.

Furthermore, all compounds of Formula (I) that exist in free base or acid form can be converted to their pharmaceutically acceptable salts by treatment with the appropriate inorganic or organic base or acid by methods known to one skilled in the art. Salts of the compounds of Formula (I) can be converted to their free base or acid form by standard techniques.

### Methods of Synthesis

The compounds described herein can be made using conventional organic syntheses and commercially available starting materials, or the methods provided herein. By way of example and not limitation, compounds of Formula (I) can be prepared as outlined in Schemes 1-4, as well as in the examples set forth herein. It should be noted that one skilled in the art would know how to modify the procedures set forth in the illustrative schemes and examples to arrive at the desired products.

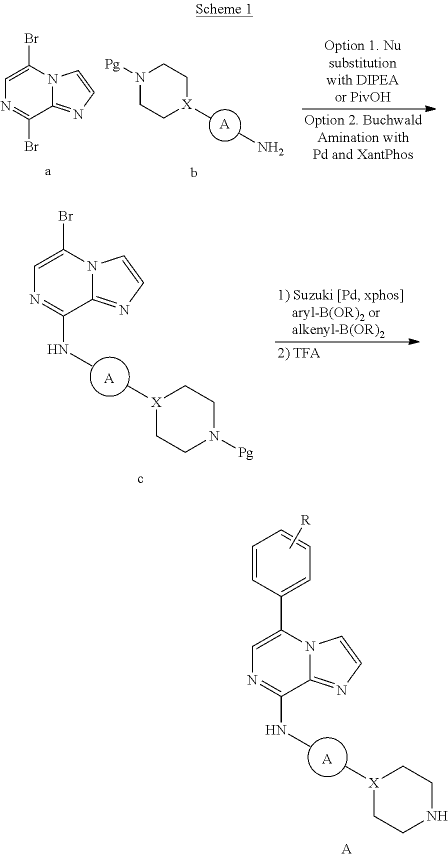

wherein Pg is a protecting group; X and Ring A are as described for Formula (I); and R is 0-5 substituents selected from C₁-C₆ alkyl, C₁-C₆ haloalkyl, C₁-C₆ alkoxy, halo, —OH, and —CN.

As outlined in Scheme 1, compounds of general formula A can be synthesized from coupling of intermediate a and intermediate b to generate intermediate C. In some instances, intermediate a and intermediate b can be coupled to form intermediate c using acid or base promoted aromatic nucleophilic substitution. Alternatively, intermediate c can be synthesized using Buchwald-Hartwig cross-coupling (Ruiz-Castillo, P. and Buchwald, S. L. Chemical Reviews, 116:12564-12649, 2016). Subsequently, Pd-catalyzed Suzuki cross-coupling (Miyaura, N. and Suzuki, A. Chemical Reviews, 95:2457-2483, 1995) of intermediate c with a suitable boronic acid or ester, followed by acid-promoted deprotection can yield compounds of general formula A. In some instances, the preparation of Compound A also involves removal of a protecting group (such as CBz) using, for example, reductive conditions.

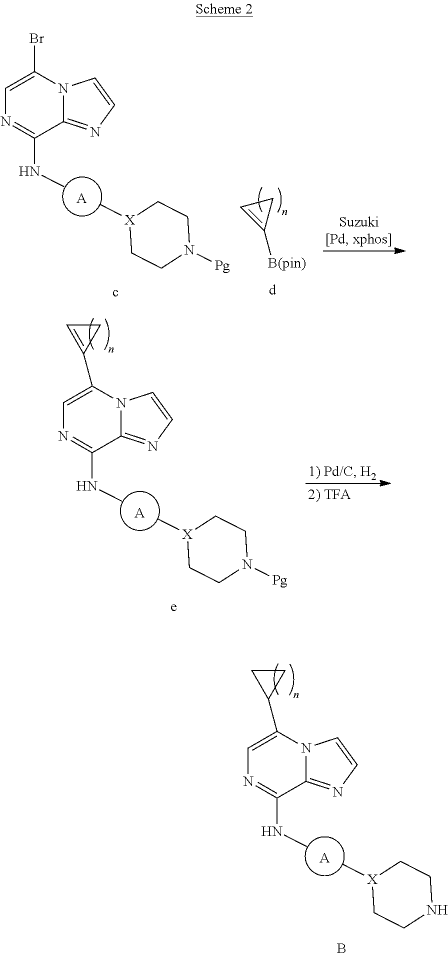

wherein Pg is a protecting group; X and Ring A are as described for Formula (I); and n is an integer of 1, 2, 3, or 4.

As outlined in Scheme 2, compounds of general formula B that contain cycloalkyl substituents on the imidazopyrazine scaffold can be synthesized from Suzuki cross-coupling of intermediate c and the corresponding alkenyl boronic acid pinacol ester (intermediate d) to generate styrenyl intermediates (e). Subsequently, palladium-catalyzed hydrogenation followed by removal of the protecting group affords compounds of general formula B.

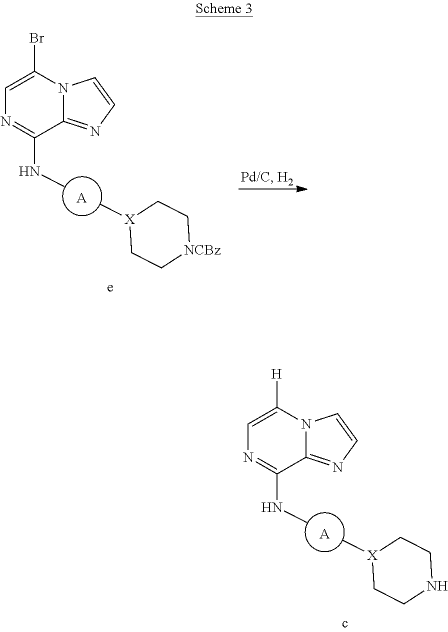

wherein X and Ring A are as described for Formula (I).

As outlined in Scheme 3, compounds of general formula C that contain an unsubstituted imidazopyrazine can be synthesized from reduction of the corresponding aryl bromide. Treatment of benzylcarbamate-protected intermediate e with palladium on carbon under an atmosphere of hydrogen gas can afford the unsubstituted imidazopyrazine exemplified by compounds of general formula C.

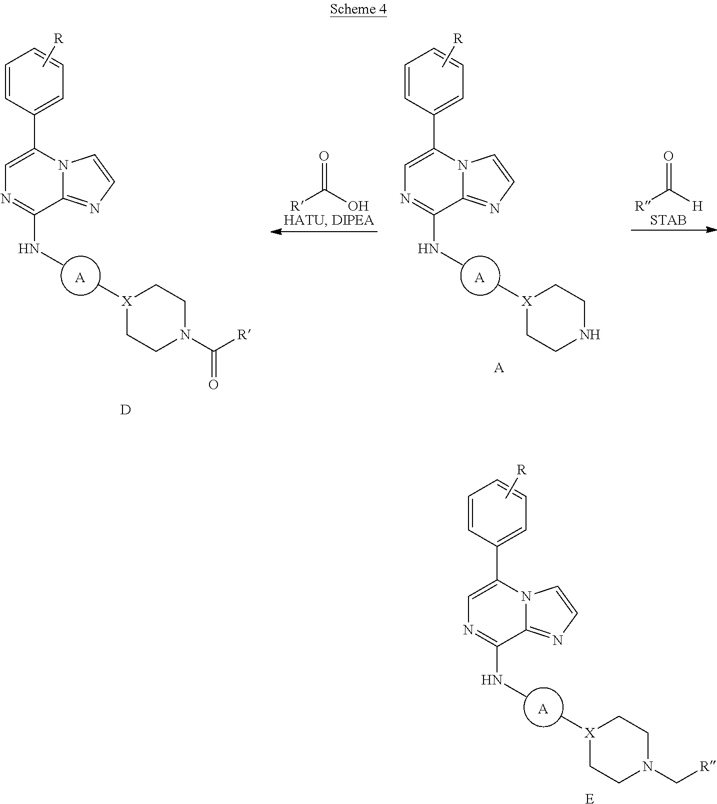

wherein R is 0-5 substituents selected from C₁-C₆ alkyl, C₁-C₆ haloalkyl, C₁-C₆ alkoxy, halo, —OH, and —CN; R′ is C₁-C₆ alkyl; R″ is C₁-C₅alkyl; and X and Ring A are as described for Formula (I).

As outlined in Scheme 4, further functionalization of compounds of general formula A via amide bond formation or reductive amination can generate compounds of general formula D or E. Treatment of compounds of general formula A with a carboxylic acid, HATU and DIPEA provides amide-capped derivatives of general formula D. Alkyl-capped compounds of general formula E can be prepared by reacting compounds of general formula A with an aldehyde and an appropriate reducing agent, such as STAB.

### Methods of Use

In some embodiments, provided herein is a method for binding IRAK3 in a subject in need thereof, the method comprising administering to the subject an effective amount of a compound of Formula (I). Binding of IRAK3 can be assessed and demonstrated by a wide variety of ways known in the art. Kits and commercially available assays can be utilized for determining whether and to what degree IRAK3 has been bound. In some embodiments, the compound of Formula (I) binds IRAK3 with an affinity of at least about 5 nM (IC₅₀). In some embodiments, the compound of Formula (I) binds IRAK3 with an affinity of about 0.5 nM, 1 nM, 1.5 nM, 2 nM, 2.5 nM, 3 nM, 3.5 nM, 4 nM, 4.5 nM, or 5 nM. In some embodiments, the compound of Formula (I) binds IRAK3 with an affinity of about 5 to 10,000 nM, about 10 to 9000 nM, about 50 to 8000 nM, about 100 to 7000 nM, about 200 to 6000 nM, about 300 to 5000 nM, about 400 to 4000 nM, about 500 to 3000 nM, about 600 to 2000 nM, about 700 to 1000 nM, or about 800 to 900 nM. In some embodiments, the compound of Formula (I) binds IRAK3 with an affinity of about 5 to 500 nM, 5 to 400 nM, 5 to 300 nM, 5 to 200 nM, or 5 to 100 nM. In some embodiments, the compound of Formula (I) binds IRAK3 with an affinity of about 5 nM, 10 nM, 15 nM, 20 nM, 25 nM, 50 nM, 100 nM, 200 nM, 300 nM, 400 nM, 500 nM, 1000 nM, 1500 nM, 2000 nM, 3000 nM, 4000 nM, 5000 nM, 6000 nM, 7000 nM, 8000 nM, 9000 nM, or 10,000 nM. In any of these embodiments, the binding affinity can be determined using the TR-FRET biochemical assay described in Example B1.

In another aspect, provided herein is a method of binding IRAK3 comprising contacting IRAK3 with an effective amount of a compound of Formula (I) or any embodiment or variation thereof.

In some embodiments, a compound of Formula (I) can be used as a ligand for heterobifunctional degraders. In some embodiments, a compound of Formula (I) is used as a ligand for heterobifunctional degraders that target IRAK3 for degradation. Such heterobifunctional degraders are useful, e.g., for treating a cancer such as bladder cancer, breast cancer, esophageal cancer, colon cancer, head and neck cancer, kidney cancer, lung cancer, pancreatic cancer, prostate cancer, melanoma, and gastric cancer. In some embodiments, the heterobifunctional degraders enhance immunity in a subject receiving a vaccine.

### Pharmaceutical Compositions and Routes of Administration

The compounds provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds disclosed herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of Formula (I) in the pharmaceutical composition may be at a level that will exercise the desired effect; for example, about 0.005 mg/kg of a subject's body weight to about 10 mg/kg of a subject's body weight in unit dosage for both oral and parenteral administration.

The dose of a compound of Formula (I) to be administered to a subject is rather widely variable and can be subject to the judgment of a health-care practitioner. In general, the compounds disclosed herein can be administered one to four times a day in a dose of about 0.001 mg/kg of a subject's body weight to about 10 mg/kg of a subject's body weight, but the above dosage may be properly varied depending on the age, body weight and medical condition of the subject and the type of administration. In one embodiment, the dose is about 0.001 mg/kg of a subject's body weight to about 5 mg/kg of a subject's body weight, about 0.01 mg/kg of a subject's body weight to about 5 mg/kg of a subject's body weight, about 0.05 mg/kg of a subject's body weight to about 1 mg/kg of a subject's body weight, about 0.1 mg/kg of a subject's body weight to about 0.75 mg/kg of a subject's body weight or about 0.25 mg/kg of a subject's body weight to about 0.5 mg/kg of a subject's body weight. In one embodiment, one dose is given per day. In any given case, the amount of the compound of Formula (I) administered will depend on such factors as the solubility of the active component, the formulation used and the route of administration.

In some embodiments, a compound of Formula (I) is administered to a subject at a dose of about 0.01 mg/day to about 750 mg/day, about 0.1 mg/day to about 375 mg/day, about 0.1 mg/day to about 150 mg/day, about 0.1 mg/day to about 75 mg/day, about 0.1 mg/day to about 50 mg/day, about 0.1 mg/day to about 25 mg/day, or about 0.1 mg/day to about 10 mg/day.

In another embodiment, provided herein are unit dosage formulations that comprise between about 0.1 mg and 500 mg, about 1 mg and 250 mg, about 1 mg and about 100 mg, about 1 mg and about 50 mg, about 1 mg and about 25 mg, or between about 1 mg and about 10 mg of a compound of Formula (I).

In a particular embodiment, provided herein are unit dosage formulations comprising about 0.1 mg or 100 mg of a compound of Formula (I).

In another embodiment, provided herein are unit dosage formulations that comprise 0.5 mg, 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a compound of Formula (I).

A compound of Formula (I) can be administered once, twice, three, four or more times daily. In a particular embodiment, doses of 100 mg or less are administered as a once daily dose and doses of more than 100 mg are administered twice daily in an amount equal to one half of the total daily dose.

A compound of Formula (I) can be administered orally for reasons of convenience. In one embodiment, when administered orally, a compound of Formula (I) is administered with a meal and water. In another embodiment, the compound of Formula (I) is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a compound of Formula (I) without an additional carrier, excipient or vehicle.

In another embodiment, provided herein are compositions comprising an effective amount of a compound of Formula (I) and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of Formula (I) with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of Formula (I) as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of Formula (I) can be delayed or prolonged by proper formulation. For example, a slowly soluble pellet of the compound of Formula (I) can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of Formula (I) in oily or emulsified vehicles that allow it to disperse slowly in the serum.

### EXEMPLARY EMBODIMENTS

The present disclosure is further described by the following embodiments. The features of each of the embodiments are combinable with any of the other embodiments where appropriate and practical.

Embodiment 1. A compound of Formula (I):

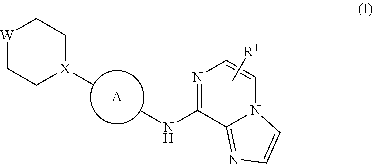

or a pharmaceutically acceptable salt thereof, wherein:
Ring A is C₆-C₁₀ aryl or 5- to 6-membered heteroaryl, wherein the heteroaryl contains 1-3 heteroatoms selected from nitrogen, oxygen, and sulfur;
R¹ is H, C₆-C₁₀ aryl, or C₃-C₆ cycloalkyl, wherein the aryl and cycloalkyl are optionally substituted by 1-5 substituents selected from C₁-C₆ alkyl, C₁-C₆ haloalkyl, C₁-C₆ alkoxy, halo, —OH, and —CN;
W is NR² or CR^{3a}R^{3b};
R² is H, C₁-C₆ alkyl, C₁-C₆ haloalkyl, or —C(O)(C₁-C₆ alkyl);
R^{3a} and R^{3b} are independently H, C₁-C₆ alkyl, C₁-C₆ haloalkyl, C₁-C₆ alkoxy, or —C(O)(C₁-C₆ alkyl); and
X is CH or N.

Embodiment 2. The compound of embodiment 1, or a pharmaceutically acceptable salt thereof, wherein:
Ring A is C₆-C₁₀ aryl.

Embodiment 3. The compound of embodiment 1, or a pharmaceutically acceptable salt thereof, wherein:
Ring A is 5- to 6-membered heteroaryl, wherein the heteroaryl contains 1-3 heteroatoms selected from nitrogen, oxygen, and sulfur.

Embodiment 4. The compound of embodiment 1 or 2, or a pharmaceutically acceptable salt thereof, wherein:
Ring A is

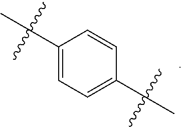 [SMILES: *c1ccc(*)cc1]

Embodiment 5. The compound of embodiment 1 or 3, or a pharmaceutically acceptable salt thereof, wherein:
Ring A is

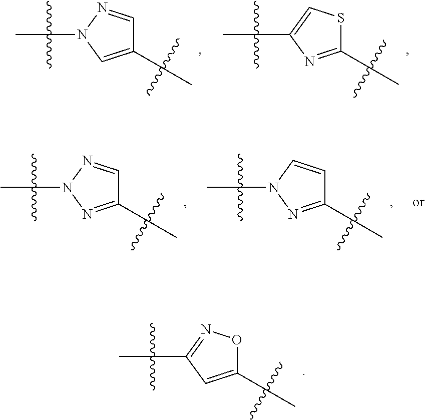 [SMILES: *c1cnn(*)c1 ; *c1csc(*)n1 ; *c1cnn(*)n1 ; *c1ccn(*)n1 ; *c1cc(*)on1]

Embodiment 6. The compound of any one of embodiments 1-5, or a pharmaceutically acceptable salt thereof, wherein R¹ is H.

Embodiment 7. The compound of any one of embodiments 1-5, or a pharmaceutically acceptable salt thereof, wherein R¹ is C₃-C₆ cycloalkyl optionally substituted by 1-5 substituents selected from C₁-C₆ alkyl, C₁-C₆ haloalkyl, C₁-C₆ alkoxy, halo, —OH, and —CN.

Embodiment 8. The compound of embodiment 7, or a pharmaceutically acceptable salt thereof, wherein R¹ is C₃-C₆ cycloalkyl.

Embodiment 9. The compound of any one of embodiments 1-5, or a pharmaceutically acceptable salt thereof, wherein R¹ is C₆-C₁₀ aryl optionally substituted by 1-5 substituents selected from C₁-C₆ alkyl, C₁-C₆ haloalkyl, C₁-C₆ alkoxy, halo, —OH, and —CN.

Embodiment 10. The compound of embodiment 9, or a pharmaceutically acceptable salt thereof, wherein R¹ is C₆-C₁₀ aryl optionally substituted by 1-5 substituents selected from C₁-C₆ alkyl.

Embodiment 11. The compound of embodiment 9, or a pharmaceutically acceptable salt thereof, wherein R¹ is C₆-C₁₀ aryl optionally substituted by 1-3 substituents selected from C₁-C₃ alkyl.

Embodiment 12. The compound of any one of embodiments 1-5 and 7-8, or a pharmaceutically acceptable salt thereof, wherein:
R1 is

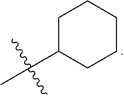 [SMILES: *C1CCCCC1]

Embodiment 13. The compound of any one of embodiments 1-5 and 9-11, or a pharmaceutically acceptable salt thereof, wherein:
R1 is

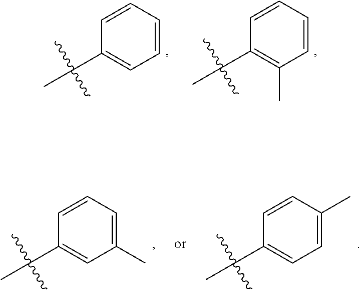

Embodiment 14. The compound of any one of embodiments 1-13, or a pharmaceutically acceptable salt thereof, wherein:
W is NR² or CR^{3a}R^{3b};
R² is H, C₁-C₆ alkyl, or —C(O)(C₁-C₃ alkyl); and
R^{3a} and R^{3b} are independently H, C₁-C₃ alkyl, or —C(O)(C₁-C₆ alkyl).

Embodiment 15. The compound of embodiment 14, or a pharmaceutically acceptable salt thereof, wherein:
W is NR²; and
R² is H, C₁-C₆ alkyl, or —C(O)(C₁-C₃ alkyl).

Embodiment 16. The compound of any one of embodiments 1-13, or a pharmaceutically acceptable salt thereof, wherein X is CH.

Embodiment 17. The compound of any one of embodiments 1-13, or a pharmaceutically acceptable salt thereof, wherein X is N.

Embodiment 18. The compound of any one of embodiments 1-15 and 16, or a pharmaceutically acceptable salt thereof, wherein:

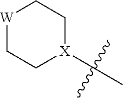

is

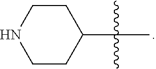 [SMILES: *C1CCNCC1]

Embodiment 19. The compound of any one of embodiments 1-15 and 17, or a pharmaceutically acceptable salt thereof, wherein:

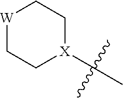

is

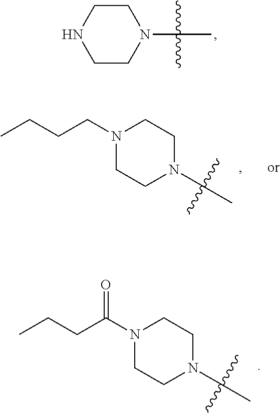 [SMILES: *N1CCNCC1 ; *N1CCN(CCCC)CC1 ; *N1CCN(C(=O)CCC)CC1]

Embodiment 20. The compound of any one of embodiments 1-5, 9-11, 13-16, or 18, or a pharmaceutically acceptable salt thereof, wherein the compound is Formula (II):

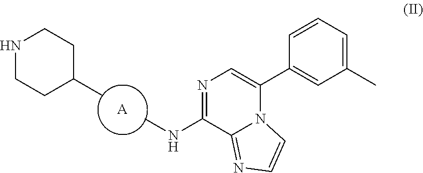

and Ring A is 5-membered heteroaryl that contains 1-3 heteroatoms selected from nitrogen, oxygen, sulfur.

Embodiment 21. The compound of any one of embodiments 1, 2, 4, 6-15, 17, or 19, or a pharmaceutically acceptable salt thereof, wherein the compound is Formula (III):

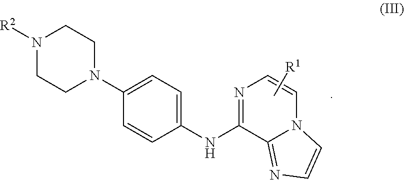

Embodiment 22. A compound selected from the compounds of Table 1 or a pharmaceutically acceptable salt thereof.

Embodiment 23. A pharmaceutical composition comprising the compound of any one of embodiments 1-22, or a pharmaceutically acceptable salt thereof, and a pharmaceutically acceptable excipient.

Embodiment 24. A method of binding Interleukin-1 Receptor-Associated Kinase 3 (IRAK3) comprising contacting IRAK3 with an effective amount of the compound of any one of embodiments 1-22, or a pharmaceutically acceptable salt thereof, or the pharmaceutical composition of embodiment 23.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

The following abbreviations may be relevant for the application.

### Abbreviations

| | |
|---|---|
| aq. | aqueous |
| Boc | tert-butyloxycarbonyl |
| Bu | butyl |
| CBz | benzyloxycarbonyl |
| DCE | dichloroethane |
| DCM | dichloromethane |
| DIEA/DIPEA/Hünig's Base | diisopropylethylamine |
| DMF | dimethyl formamide |
| DMSO | dimethyl sulfoxide |
| EGTA | ethylene glycol-bis(2-aminoethylether)-N,N,N′,N′-tetraacetic |
| | acid |
| equiv. | equivalent |
| ESI | electrospray ionization |
| Et | ethyl |
| EtOAc | ethyl acetate |
| EtOH | ethanol |
| FA | formic acid |
| h | hour |
| HATU | O-(7-azabenzotriazol-1-yl)-N,N,N′,N′-tetramethyluronium |
| | hexafluorophosphate |
| HCl | hydrochloric acid |
| HEPES | 4-(2-hydroxyethyl)-1-piperazineethanesulfonic acid |
| HPLC | high performance liquid chromatography |
| i-Pr | isopropyl |
| LC/MS, LCMS | liquid chromatography mass spectrometry |
| Me | methyl |
| MeCN | acetonitrile |
| MeOH | methanol |
| min | minute |
| MS | mass spectrometry |
| NaBH(OAc)₃ / STAB | sodium triacetoxyborohydride |
| NMR | nuclear magnetic resonance |
| Pd(PPh₃)₄ | palladium tetrakis |
| Pd/C | palladium on carbon |
| Pd₂(dba)₃ | tris(dibenzylideneacetone)dipalladium(0) |
| PdCl₂(dppf) | [1,1′-bis(diphenylphosphino)-ferrocene]dichloropalladium(II) |
| pin | pinacolato |
| PivOH | pivalic acid |
| rt | retention time |
| sat. | saturated |
| SiO₂ | silica oxide |
| t-BuOK | potassium tert-butoxide |
| TEA | triethylamine |
| TFA | trifluoroacetic acid |
| THF | tetrahydrofuran |
| TLC | thin layer chromatography |
| TR-FRET | time-resolved fluorescence resonance energy transfer |
| Xantphos | 4,5-bis(diphenylphosphino)-9,9-dimethylxanthene |
| Xphos | 2-dicyclohexylphosphino-2′,4′,6′-triisopropylbiphenyl |
| Xphos Pd G3 | 2-dicyclohexylphosphino-2′,4′,6′-triisopropyl-1,1′-biphenyl)[2- |
| | (2′-amino-1,1′-biphenyl)]palladium(II) methanesulfonate |

### SYNTHETIC EXAMPLES

### Example S1. Synthesis of N-(4-(Piperazin-1-yl)phenyl)imidazo[1,2-a]pyrazin-8-amine (1)

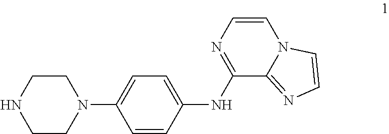 [SMILES: c1cn2ccnc2c(Nc2ccc(N3CCNCC3)cc2)n1]

Synthesis of Benzyl 4-(4-((5-bromoimidazo[1,2-a]pyrazin-8-yl)amino)phenyl)piperazine-1-carboxylate. A 40 mL vial equipped with stir bar was charged with 6,8-dibromoimidazo[1,2-a]pyrazine (0.89 g, 3.2 mmol) and benzyl 4-(4-aminophenyl)piperazine-1-carboxylate (1.1 equiv, 1.1 g, 3.5 mmol). Then iso-propanol (10.7 mL, 0.3 M) and N,N-diisopropylethylamine (2.0 equiv., 1.1 mL, 6.4 mmol) were added to the vial. The vial was then sealed with a septum and mixed by vortex. The reaction vessel was heated to 65° C. and allowed to stir for 72 h. The reaction was allowed to cool to room temperature. The reaction mixture was poured into water (20 mL) and mixed by vortex. Initially the crude reaction mixture contained a brown sludge, which solidified upon sufficient mixing with water. The mixture was then filtered, and the filter cake was washed with water (20 mL) and diethyl ether (20 mL). The resulting solid was collected and dried on high vac, affording the product, benzyl 4-(4-((5-bromoimidazo[1,2-a]pyrazin-8-yl)amino)phenyl)piperazine-1-carboxylate (1.37 g, 2.7 mmol, 84% yield), as a light brown solid. LC/MS Method 2: MS (ESI) [M+H]⁺ 508.4, rt: 1.71 min.

Synthesis of N-(4-(Piperazin-1-yl)phenyl)imidazo[1,2-a]pyrazin-8-amine. A 20 mL vial containing benzyl 4-[4-[(6-bromoimidazo[1,2-a]pyrazin-8-yl)amino]phenyl]piperazine-1-carboxylate (50.0 mg, 0.099 mmol) was charged with 10% Pd/C (10.5 mg, 10 mol %). A stir bar was then added, and the vial was sealed with a septa cap. The solids were suspended in methanol (1.0 mL, 0.1 M). The vial was then evacuated and refilled by a balloon containing H₂. This process was repeated 5 times. The reaction was then stirred at 22° C. for 72 h. The vial was purged with N₂ gas to remove the traces of H₂ gas. The crude reaction mixture was then filtered through a plug of celite, rinsing carefully with excess methanol. LCMS analysis of the crude reaction mixture revealed partial conversion of the starting material to the desired product. The filtrate was then concentrated and purified by reverse phase column chromatography (5% to 100% water/MeCN with 0.1% TFA) affording the product, N-(4-(piperazin-1-yl)phenyl)imidazo[1,2-a]pyrazin-8-amine (40 mg, 0.0985 mmol, 98% yield), as a yellow solid after lyophilization. LC/MS Method 2: MS (ESI) [M+H]⁺ 295.4, rt: 0.18 min. 1H NMR (400 MHz, methanol-d₄) δ ppm 3.32-3.43 (m, 4H) 3.45-3.54 (m, 4H) 7.15 (d, J=5.5 Hz, 1H) 7.17-7.23 (m, 2H) 7.50 (d, J=8.1 Hz, 2H) 7.83 (s, 1H) 7.99 (d, J=5.4 Hz, 1H) 8.07 (d, J=1.0 Hz, 1H).

### Example S2. Synthesis of 5-Phenyl-N-(4-(piperazin-1-yl)phenyl)imidazo[1,2-a]pyrazin-8-amine (2)

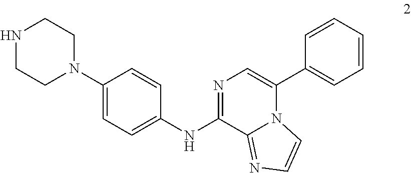 [SMILES: c1ccc(-c2cnc(Nc3ccc(N4CCNCC4)cc3)c3nccn23)cc1]

Synthesis of tert-Butyl 4-(4-((5-bromoimidazo[1,2-a]pyrazin-8-yl)amino)phenyl)piperazine-1-carboxylate. tert-butyl 4-(4-aminophenyl)piperazine-1-carboxylate (604.3 mg, 2.18 mmol) and DIPEA (0.38 mL, 2.18 mmol) were added to a solution of 5,8-dibromoimidazo[1,2-a]pyrazine (402.2 mg, 1.45 mmol) in ethanol (2.69 mL). The reaction was heated to 80° C. and stirred overnight. LCMS showed that the reaction went to 94% conversion and the reaction was stopped. Ethanol was removed in vacuo and the product was taken up in DCM. The resulting solution was washed with water, dried over MgSO₄, and concentrated in vacuo. The residue was purified by reverse phase column chromatography (5% to 100% MeOH in water with 0.1% formic acid), affording the desired product, tert-butyl 4-(4-((5-bromoimidazo[1,2-a]pyrazin-8-yl)amino)phenyl)piperazine-1-carboxylate (443 mg, 0.935 mmol, 64% yield). LC/MS Method 1: MS (ESI) [M]⁺ 473.2, rt: 1.479 min.

Synthesis of tert-Butyl 4-(4-((5-phenylimidazo[1,2-a]pyrazin-8-yl)amino)phenyl)piperazine-1-carboxylate. tert-butyl 4-[4-[(5-bromoimidazo[1,2-a]pyrazin-8-yl)amino]phenyl]piperazine-1-carboxylate (80.0 mg, 0.1700 mmol), phenylboronic acid (24.73 mg, 0.20 mmol), 1,4-dioxane (3.5 mL) and aq. NaHCO₃ sat. (1.0 mL, 0.170 mmol) were placed in a sealed vial. The solution was sparged with nitrogen for 5 min then Pd(PPh₃)₄ (9.76 mg, 0.010 mmol) was added, and the mixture was sparged with nitrogen for 5 min. The solution was heated to 95° C. for 16 h. The following day, the solution had turned orange. TLC analysis (eluting 3 times with 40% EtOAc in heptane) showed that the reaction did not reach completion, so another 0.05 eq of Pd(PPh₃)₄ was added and the reaction mixture was stirred at 95° C. for an additional 4 h. The reaction was stopped, quenched with water, and extracted with EtOAc (2×). The organic layers were washed with brine, dried with MgSO₄, and concentrated in vacuo. The residue was purified by reverse phase column chromatography (0% to 100% MeOH in water with 0.1% formic acid), affording the desired product, tert-butyl 4-(4-((5-phenylimidazo[1,2-a]pyrazin-8-yl)amino)phenyl)piperazine-1-carboxylate (58.7 mg, 0.124 mmol, 73% yield), as a tan solid. LC/MS Method 1: MS (ESI) [M+H]⁺ 471.4, rt: 1.454 min.

Synthesis of 5-Phenyl-N-(4-(piperazin-1-yl)phenyl)imidazo[1,2-a]pyrazin-8-amine. To a solution of tert-butyl 4-[4-[(5-phenylimidazo[1,2-a]pyrazin-8-yl)amino]phenyl]piperazine-1-carboxylate (58.7 mg, 0.120 mmol) in methanol (1.2474 mL) at room temperature was added HCl in dioxane (477.23 μL, 1.91 mmol). The resulting solution was stirred at room temperature for 3 h and 30 min. The mixture was concentrated on the rotovap and the residue was purified by reverse phase column chromatography (5% to 100% MeOH in water with 0.1% formic acid) affording the product as an impure yellow oil. This oil was repurified by reverse phase column chromatography (5% to 100% MeOH in water with pH 10), affording the product, 5-phenyl-N-(4-(piperazin-1-yl)phenyl)imidazo[1,2-a]pyrazin-8-amine (17.2 mg, 0.046 mmol, 37% yield) as a yellow solid after lyophilization. MS (ESI) [M+H]⁺ 371.1. ¹H NMR (400 MHz, DMSO-d₆) δ ppm 2.78-2.89 (m, 4H), 2.93-3.06 (m, 4H), 3.30-3.32 (m, 1H), 6.91 (br d, J=8.9 Hz, 2H), 7.41 (s, 1H), 7.49-7.62 (m, 3H), 7.64-7.72 (m, 3H), 7.83-7.92 (m, 3H), 9.41 (s, 1H).

### Example S3. Synthesis of 5-Cyclohexyl-N-(4-(piperazin-1-yl)phenyl)imidazo[1,2-a]pyrazin-8-amine (3)

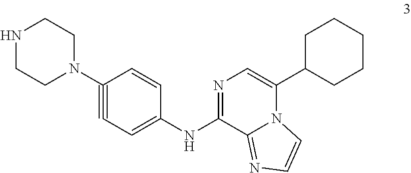

Synthesis of tert-Butyl 4-(4-((5-(cyclohex-1-en-1-yl)imidazo[1,2-a]pyrazin-8-yl)amino)phenyl)piperazine-1-carboxylate. A 0.5-2 mL microwave reaction tube equipped with stir bar was charged with tert-8butyl 4-[4-[(5-bromoimidazo[1,2-a]pyrazin-8-yl)amino]phenyl]piperazine-1-carboxylate (75 mg, 0.16 mmol), cyclohexen-1-ylboronic (39.9 mg, 0.32 mmol), and XPhos-Pd-G3 (10.0 mg, 0.012 mmol) and Xphos (5.7 mg, 0.012 mmol). The reaction vessel was then sealed with a septum and purged with nitrogen. 1,4-Dioxane (0.8 mL, 0.2 M) and 2 M aqueous sodium carbonate (240 μL, 0.295 mmol) were then added to the reaction vessel via syringe. The reaction vessel was then sealed with a microwave cap and then mixed by vortex. The vessel was then heated at 135° C. under microwave irradiation for 45 min. After this time, the reaction was then allowed to cool to 22° C. and filtered through a pad of celite. The filter cake was then washed with methanol. LCMS analysis of the crude reaction mixture revealed formation of the desired product. The reaction mixture was then concentrated and purified by reverse phase column chromatography (15% to 100% MeCN in water with 0.1% TFA). The fractions were than concentrated and dried in vacuo, affording the product, tert-Butyl 4-(4-((5-(cyclohex-1-en-1-yl)imidazo[1,2-a]pyrazin-8-yl)amino)phenyl)piperazine-1-carboxylate (60.7 mg, 0.128 mmol, 80% yield), as an off-white solid. LC/MS Method 2: MS (ESI) [M+H]⁺ 475.4, rt: 1.76 min.

Synthesis of 5-Cyclohexyl-N-(4-(piperazin-1-yl)phenyl)imidazo[1,2-a]pyrazin-8-amine. tert-Butyl 4-[4-[[5-(cyclohexen-1-yl)imidazo[1,2-a]pyrazin-8-yl]amino]phenyl]piperazine-1-carboxylate (39.7 mg, 0.084 mmol) and palladium hydroxide on carbon powder (6.0 mg, 10 mol %) were added to a 20 mL vial equipped with stir bar. Methanol (0.03 M, 2.8 mL) was then added, and the vial was sealed with a septa cap. The reaction vessel was then evacuated and backfilled with hydrogen (3×). The reaction was then allowed to stir overnight under an atmosphere of hydrogen. The following morning the reaction was filtered through a pad of celite and washed with methanol. LCMS analysis of the crude reaction mixture revealed formation of the desired product, with traces of the unsaturated isomer remaining. The reaction mixture was purified by reverse phase column chromatography (15% to 100% MeCN in water with 0.1% TFA). The collected fractions were concentrated and immediately treated with 1:1 TFA/DCM. LCMS analysis of the crude reaction mixture revealed full deprotection of the piperazine. The unpurified residue was mass-directed HPLC (5% to 100% MeCN in water with 0.1% formic acid), affording the product, 5-cyclohexyl-N-(4-(piperazin-1-yl)phenyl)imidazo[1,2-a]pyrazin-8-amine (15.4 mg, 0.041 mmol, 48% yield), as a light-yellow solid after lyophilization. MS (ESI) [M+H]⁺ 371.1. ¹H NMR (400 MHz, methanol-d₄) δ ppm 1.45-1.63 (m, 4H) 1.92 (br d, J=12.8 Hz, 2H) 2.14 (br d, J=12.1 Hz, 2H) 2.95 (br t, J=11.2 Hz, 1H) 3.37-3.48 (m, 8H) 6.97 (s, 1H) 7.15 (m, 2H) 7.57 (m, 2H) 7.79 (s, 1H) 8.13 (s, 1H).

### Example S4. Synthesis of N-(4-(Piperazin-1-yl)phenyl)-5-(o-tolyl)imidazo[1,2-a]pyrazin-8-amine (4)

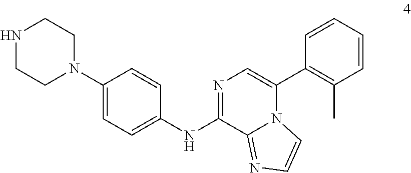 [SMILES: Cc1ccccc1-c1cnc(Nc2ccc(N3CCNCC3)cc2)c2nccn12]

Synthesis of tert-Butyl 4-(4-((5-(o-tolyl)imidazo[1,2-a]pyrazin-8-yl)amino)phenyl)piperazine-1-carboxylate. A dry seal tube was charged with tert-butyl 4-[4-[(5-bromoimidazo[1,2-a]pyrazin-8-yl)amino]phenyl]piperazine-1-carboxylate (110. mg, 0.230 mmol) and o-tolylboronic acid (38.78 mg, 0.290 mmol) at room temperature. Then, 1,4-dioxane (2.7463 mL) and saturated aq. NaHCO₃ (1.2 mL) were added, and the mixture was sparged with nitrogen for 20 minutes. Pd(PPh₃)₄ (13.94 mg, 0.010 mmol) was added in one portion, and sparging with nitrogen was continued for 10 minutes. The tube was sealed and placed in an oil bath pre-equilibrated to 95° C. for 2 hours. Then, the reaction mixture was diluted with 20 mL of sat. NaHCO₃ and extracted with EtOAc (3×10 mL). Combined organics were washed with brine, dried over MgSO₄, filtered, and concentrated. The residue was purified by reverse phase column chromatography (5% to 100% MeOH in water with 0.1% formic acid, affording the desired product, tert-butyl 4-(4-((5-(o-tolyl)imidazo[1,2-a]pyrazin-8-yl)amino)phenyl)piperazine-1-carboxylate (97 mg, 0.184 mmol, 79% yield) as a tan solid. LC/MS Method 1: MS (ESI) [M+H]⁺ 485.4, rt: 1.464 min.

Synthesis of N-(4-(Piperazin-1-yl)phenyl)-5-(o-tolyl)imidazo[1,2-a]pyrazin-8-amine. To a solution of tert-butyl 4-[4-[[5-(o-tolyl)imidazo[1,2-a]pyrazin-8-yl]amino]phenyl]piperazine-1-carboxylate (97. mg, 0.20 mmol) in methanol (1.2535 mL) at room temperature was added HCl in dioxane (765.79 μL, 3.06 mmol). The resulting solution was stirred at room temperature. After 4 hours, HPLC analysis showed approx. 95% conversion. The solution was left to stir for another hour and then the resulting suspension was concentrated on the rotovap and the residue was purified by reverse phase column chromatography (5% to 100% MeOH in water with 0.1% formic acid) affording the product as an impure light yellow solid. This material was repurified by reverse phase column chromatography (5% to 100% MeOH in water with pH 10), affording the product, N-(4-(piperazin-1-yl)phenyl)-5-(o-tolyl)imidazo[1,2-a]pyrazin-8-amine (19.6 mg, 0.05 mmol, 25% yield), as an off-white solid after lyophilization. [M+H]⁺ 385.2. ¹H NMR (400 MHz, DMSO-d₆) δ ppm 2.13 (s, 3H), 2.52-2.55 (m, 1H), 2.80-2.88 (m, 4H), 2.96-3.04 (m, 4H), 6.91 (d, J=9.0 Hz, 2H), 7.29 (s, 2H), 7.34-7.50 (m, 4H), 7.60 (s, 1H), 7.87 (d, J=9.0 Hz, 2H), 9.38 (s, 1H).

### Example S5. Synthesis of N-(4-(Piperazin-1-yl)phenyl)-5-(m-tolyl)imidazo[1,2-a]pyrazin-8-amine (5)

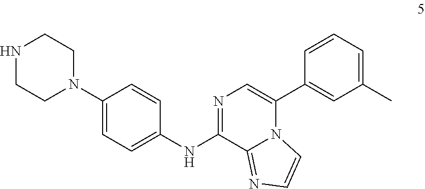 [SMILES: Cc1cccc(-c2cnc(Nc3ccc(N4CCNCC4)cc3)c3nccn23)c1]

Synthesis of tert-Butyl 4-(4-((5-(m-tolyl)imidazo[1,2-a]pyrazin-8-yl)amino)phenyl)piperazine-1-carboxylate. In a microwave vial, a mixture of 2 M aq. Na₂CO₃ (1.01 mL, 2.03 mmol) and m-tolylboronic acid (91.91 mg, 0.680 mmol) in 1,4-dioxane (5 mL) and tert-butyl 4-[4-[(5-bromoimidazo[1,2-a]pyrazin-8-yl)amino]phenyl]piperazine-1-carboxylate (320 mg, 0.680 mmol) was flushed with N₂ for 10 min, then Pd(Ph₃P)₄ (78.12 mg, 0.070 mmol) was added and the suspension was flushed for another 10 min with nitrogen. The reaction was heated to 100° C. and irradiated in a microwave reactor for 1 h. The reaction was then partitioned between EtOAc/H₂O and extracted with EtOAc (2×). The organic layer was then washed with H₂O, brine, dried over Na₂SO₄. The reaction was then concentrated directly onto silica gel and purified by normal phase chromatography (30% to 100% EtOAc in heptane), affording tert-butyl 4-(4-((5-(m-tolyl)imidazo[1,2-a]pyrazin-8-yl)amino)phenyl)piperazine-1-carboxylate (80% pure, 198 mg, 0.327 mmol, 48% yield), as an orange solid. LC/MS Method 1: MS (ESI) [M+H]⁺ 485.4, rt: 1.504 min.

Synthesis of N-(4-(Piperazin-1-yl)phenyl)-5-(m-tolyl)imidazo[1,2-a]pyrazin-8-amine. Art solution of tert-butyl 4-[4-[[5-(m-tolyl)imidazo[1,2-a]pyrazin-8-yl]amino]phenyl]piperazine-1-carboxylate (580 mg, 1.2 mmol) in DCM (5 mL) was treated with TFA (3.0 mL, 11.76 mmol). The reaction was allowed to stir at for 4 h. The reaction mixture was concentrated to dryness, taken up in DCM and washed with sat. aq. NaHCO₃ (1×). The aqueous layer was then back-extracted with DCM (2×) and organic layers were combined and concentrated. The resulting residue was purified by reverse phase column chromatography (5% to 100% MeCN in water with 0.1% formic acid) affording the product, N-(4-(piperazin-1-yl)phenyl)-5-(m-tolyl)imidazo[1,2-a]pyrazin-8-amine (256.6 mg, 0.666 mmol, 56% yield), as an orange solid after lyophilization. MS (ESI) [M+H]⁺=385.2. ¹H NMR (400 MHz, DMSO-d₆) δ ppm 2.42 (s, 3H), 3.23 (br s, 4H), 3.31-3.59 (m, 4H), 7.10 (d, J=8.8 Hz, 2H), 7.38-7.43 (m, 1H), 7.44-7.60 (m, 4H), 7.79 (br d, J=7.6 Hz, 2H), 8.08-8.16 (m, 2H), 9.34 (br s, 2H), 10.93 (br s, 1H).

### Example S6. Synthesis of N-(4-(Piperazin-1-yl)phenyl)-5-(p-tolyl)imidazo[1,2-a]pyrazin-8-amine (6)

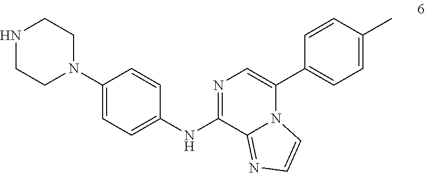 [SMILES: Cc1ccc(-c2cnc(Nc3ccc(N4CCNCC4)cc3)c3nccn23)cc1]

Synthesis of tert-Butyl 4-(4-((5-(p-tolyl)imidazo[1,2-a]pyrazin-8-yl)amino)phenyl)piperazine-1-carboxylate. A dry seal tube was charged with tert-butyl 4-[4-[(5-bromoimidazo[1,2-a]pyrazin-8-yl)amino]phenyl]piperazine-1-carboxylate (64.8 mg, 0.140 mmol) and p-tolylboronic acid (22.6 mg, 0.170 mmol) at room temperature. Then, 1,4-dioxane (3 mL) and saturated aqueous NaHCO₃ (1 mL) were added, and the mixture was sparged with nitrogen for 8 minutes. Pd(PPh₃)₄ (8.4 mg, 0.010 mmol) was added in one portion, and sparging with nitrogen was continued for 10 minutes. The tube was sealed and placed in an oil bath pre-equilibrated to 95° C. The mixture was heated at that temperature overnight. LCMS analysis revealed that the reaction went to completion. The reaction flask was taken out of the oil bath and the mixture was allowed to cool to room temperature. The reaction mixture was diluted with 5 mL of water, and the mixture was extracted with EtOAc (10 mL). The phases were separated, and the aqueous layer was extracted with EtOAc (2×10 mL). Combined organics were washed with brine, dried over MgSO₄, filtered, and concentrated. The residue was purified by reverse phase column chromatography (5% to 100% MeOH in water with 0.1% formic acid) affording the product, tert-butyl 4-(4-((5-(p-tolyl)imidazo[1,2-a]pyrazin-8-yl)amino)phenyl)piperazine-1-carboxylate (57 mg, 0.117 mmol, 86% yield), as an orange solid. LC/MS Method 1: MS (ESI) [M+H]⁺ 485.4, rt: 1.507 min.

Synthesis of N-(4-(Piperazin-1-yl)phenyl)-5-(p-tolyl)imidazo[1,2-a]pyrazin-8-amine. To a solution of tert-butyl 4-[4-[[5-(p-tolyl)imidazo[1,2-a]pyrazin-8-yl]amino]phenyl]piperazine-1-carboxylate (57.0 mg, 0.120 mmol) in methanol (1 mL) at room temperature was added HCl in dioxane (450.0 μL, 1.8 mmol). The resulting solution was stirred at room temperature overnight. The following morning, HPLC analysis showed the reaction went to completion. The reaction mixture was concentrated and purified by reverse phase column chromatography (5% to 100% MeOH in water with 0.1% formic acid), affording the product as an impure light-yellow solid. This solid was repurified by reverse phase column chromatography (5% to 100% MeOH in water with pH 10 buffer), affording the product, N-(4-(piperazin-1-yl)phenyl)-5-(p-tolyl)imidazo[1,2-a]pyrazin-8-amine (28 mg, 0.074 mmol, 62% yield), as a yellow solid after lyophilization. MS (ESI) [M+H]⁺=385.2. ¹H NMR (400 MHz, DMSO-d₆) δ ppm 2.40 (s, 3H), 2.52-2.53 (m, 1H), 2.81-2.86 (m, 4H), 2.97-3.02 (m, 4H), 6.90 (d, J=9.3 Hz, 2H), 7.35-7.41 (m, 3H), 7.57 (d, J=8.1 Hz, 2H), 7.65 (d, J=1.0 Hz, 1H), 7.83-7.89 (m, 3H), 9.37 (s, 1H).

### Example S7. Synthesis of N-(4-(Piperazin-1-yl)phenyl)-6-(m-tolyl)imidazo[1,2-a]pyrazin-8-amine (7)

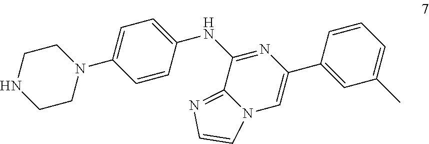 [SMILES: Cc1cccc(-c2cn3ccnc3c(Nc3ccc(N4CCNCC4)cc3)n2)c1]

Synthesis of tert-Butyl 4-(4-((6-bromoimidazo[1,2-a]pyrazin-8-yl)amino)phenyl)piperazine-1-carboxylate. 6,8-Dibromoimidazo[1,2-a]pyrazine (1.0 g, 3.6 mmol) and 1-boc-4-(4-aminophenyl)piperazine (1.5 g, 1.5 equiv., 5.4 mmol) were added to a 20 mL vial equipped with stir bar. The solids were then dissolved in ethanol (7 mL, 0.5 M) and the vial was then sealed with a septa cap. The reaction mixture was then allowed to stir for ~5 mins, prior to the addition of N,N-diisopropylethylamine (1.3 mL, 2.0 equiv., 7.2 mmol). The reaction was then warmed to 80° C. and allowed to stir over the weekend. LCMS analysis of the crude reaction mixture revealed full conversion to the desired product. The reaction mixture was concentrated onto silica gel and purified by normal phase column chromatography (0% to 10% MeOH in DCM), affording the product, (0.9 g, 1.91 mmol, 53% yield), as a dark yellow solid. LC/MS Method 2: MS (ESI) [M]⁺ 473.4, rt: 1.68 min.

Synthesis of tert-Butyl 4-(4-((6-(m-tolyl)imidazo[1,2-a]pyrazin-8-yl)amino)phenyl)piperazine-1-carboxylate. A microwave tube was charged with tert-butyl 4-[4-[(6-bromoimidazo[1,2-a]pyrazin-8-yl)amino]phenyl]piperazine-1-carboxylate (80. mg, 0.170 mmol) and m-tolylboronic acid (27.66 mg, 0.200 mmol) at rt. Then, 1,4-dioxane (3 mL) and saturated aqueous NaHCO₃ (1 mL) were added, and the mixture was sparged with nitrogen for 5 min. Pd(PPh₃)₄ (11.54 mg, 0.010 mmol) was added in one portion, and sparging with nitrogen was continued for 5 min. The reaction mixture was heated to 100° C. and subjected to microwave irradiation for 1 h. After cooling to rt, LCMS showed complete conversion into the desired product. The reaction mixture was diluted with 5 mL of water, and the mixture was extracted with EtOAc. The phases were separated, and the aqueous layer was extracted with EtOAc (2×). The combined organics were washed with brine, dried over MgSO₄, filtered, and concentrated. The residue was purified by reverse phase column chromatography (5% to 100% MeOH in water with 0.1% formic acid), affording the product, tert-butyl 4-(4-((6-(m-tolyl)imidazo[1,2-a]pyrazin-8-yl)amino)phenyl)piperazine-1-carboxylate (59 mg, mmol, 72% yield), as a light-yellow. LC/MS Method 1: MS (ESI) [M+H]⁺ 485.4, rt: 1.536 min.

Synthesis of N-(4-(Piperazin-1-yl)phenyl)-6-(m-tolyl)imidazo[1,2-a]pyrazin-8-amine. To a solution of tert-butyl 4-[4-[[6-(m-tolyl)imidazo[1,2-a]pyrazin-8-yl]amino]phenyl]piperazine-1-carboxylate (59.0 mg, 0.120 mmol) in DCM was added 4 M HCl in dioxane (0.49 mL, 1.95 mmol) at rt. The resulting solution was stirred at rt, overnight. LCMS showed complete conversion. The mixture was concentrated to dryness and solubilized in 25 mL of 1:1 MeOH/DCM. This solution was filtered through a pad of NaHCO₃ and concentrated to dryness. The crude residue was purified by reverse phase column chromatography (5% to 100% MeOH in water with 0.1% formic acid), affording the product, N-(4-(piperazin-1-yl)phenyl)-6-(m-tolyl)imidazo[1,2-a]pyrazin-8-amine (20.6 mg, 0.054 mmol, 44% yield), as a yellow solid after lyophilization. MS (ESI) [M+H]⁺ 385.2. ¹H NMR (400 MHz, DMSO-d₆) δ ppm 2.37-2.43 (m, 3H), 2.51-2.54 (m, 1H), 2.78-2.90 (m, 4H), 2.96-3.08 (m, 4H), 6.95 (d, J=9.0 Hz, 2H), 7.19 (d, J=7.6 Hz, 1H), 7.37 (t, J=7.6 Hz, 1H), 7.62 (d, J=1.0 Hz, 1H), 7.73-7.86 (m, 2H), 7.91-8.04 (m, 3H), 8.55 (s, 1H), 9.44 (s, 1H).

### Example S8. Synthesis of 1-(4-(4-((5-(m-Tolyl)imidazo[1,2-a]pyrazin-8-yl)amino)phenyl)piperazin-1-yl)butan-1-one (8)

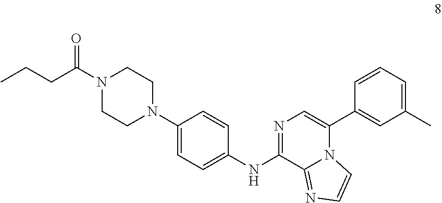 [SMILES: CCCC(=O)N1CCN(c2ccc(Nc3ncc(-c4cccc(C)c4)n4ccnc34)cc2)CC1]

HATU (13.6 mg, 1.0 equiv) was added to a 2 mL vial equipped with stir bar. DMF (120 μL, 0.3 M) was then added followed by butyric acid (3.3 μL, 1.0 equiv). DIPEA (13.2 μL, 4.0 equiv.) was then added. The resulting reaction mixture was allowed to stir for ~5 min, prior to the addition of N-(4-(Piperazin-1-yl)phenyl)-5-(m-tolyl)imidazo[1,2-a]pyrazin-8-amine; hydrochloride (15 mg, 0.037 mmol) The vial was then sealed with a cap and allowed to stir overnight at room temp. LCMS analysis revealed ~66% conversion to the desired product. The reaction mixture was then purified by reverse phase column chromatography (15% to 100% MeCN in water with 0.1% TFA), affording the product, 1-(4-(4-((5-(m-tolyl)imidazo[1,2-a]pyrazin-8-yl)amino)phenyl)piperazin-1-yl)butan-1-one (8.2 mg, 0.0179 mmol, 50% yield) as a yellow solid after lyophilization. MS (ESI) [M+H]⁺ 455.6. ¹H NMR (400 MHz, methanol-d₄) δ ppm 1.00 (t, J=7.4 Hz, 3H), 1.67 (sxt, J=7.5 Hz, 2H), 2.42-2.48 (m, 5H), 3.23-3.35 (m, 4H) (overlaps with d₄-MeOH), 3.76 (dt, J=10.6, 5.2 Hz, 4H), 7.07 (s, 1H), 7.20 (d, J=8.9 Hz, 2H), 7.41-7.52 (m, 6H), 7.83 (s, 1H), 7.96 (s, 1H).

### Example S9. Synthesis of N-(4-(4-Butylpiperazin-1-yl)phenyl)-5-(m-tolyl)imidazo[1,2-a]pyrazin-8-amine (9)

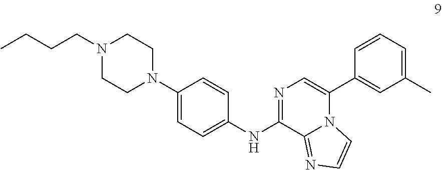 [SMILES: CCCCN1CCN(c2ccc(Nc3ncc(-c4cccc(C)c4)n4ccnc34)cc2)CC1]

N-(4-(piperazin-1-yl)phenyl)-5-(m-tolyl)imidazo[1,2-a]pyrazin-8-amine; hydrochloride (15 mg, 0.036 mmol) was added to a 1-dram vial equipped with stir bar. DCE (42 μL) was then added, followed by triethylamine (8 μL, 1.5 equiv.) The resulting solution was allowed to stir for ~15 min. At this point, sodium triacetoxyborohydride (11.3 mg, 1.5 equiv.) was added followed by a 100 μL solution of DCE containing butyraldehyde (3.2 μL, 1.0 equiv.) and acetic acid (6.2 μL, 3.0 equiv). The resulting mixture was allowed to stir at room temp. overnight. LCMS analysis of the reaction mixture revealed ~88% conversion to the desired product. The reaction mixture was then purified by reverse phase column chromatography (15% to 100% MeCN in water with 0.1% TFA), affording the product, N-(4-(4-butylpiperazin-1-yl)phenyl)-5-(m-tolyl)imidazo[1,2-a]pyrazin-8-amine (8.1 mg, 0.018 mmol, 51% yield), as a yellow solid after lyophilization. MS (ESI) [M+H]⁺ 441.6. ¹H NMR (400 MHz, methanol-d₄) δ ppm 1.03 (t, J=7.4 Hz, 3H), 1.47 (sxt, J=7.5 Hz, 2H), 1.73-1.83 (m, 2H), 2.45 (s, 3H), 3.11 (br d, J=11.5 Hz, 2H), 3.18-3.28 (m, 4H), 3.70 (br d, J=11.0 Hz, 2H), 3.90 (br d, J=11.4 Hz, 2H), 7.17 (d, J=8.9 Hz, 2H), 7.21 (s, 1H), 7.38-7.50 (m, 4H), 7.66 (d, J=8.9 Hz, 2H), 7.76 (s, 1H), 7.92 (s, 1H).

### Example S10. Synthesis of N-(1-(Piperidin-4-yl)-1H-pyrazol-4-yl)-5-(m-tolyl)imidazo[1,2-a]pyrazin-8-amine (10)

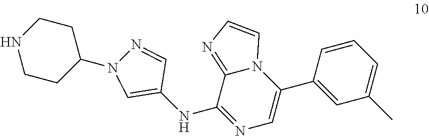 [SMILES: Cc1cccc(-c2cnc(Nc3cnn(C4CCNCC4)c3)c3nccn23)c1]

Synthesis of tert-Butyl 4-(4-((5-bromoimidazo[1,2-a]pyrazin-8-yl)amino)-1H-pyrazol-1-yl) piperidine-1-carboxylate. 5,8-Dibromoimidazo[1,2-a]pyrazine (500 mg, 1.8 mmol), tert-butyl 4-(4-aminopyrazol-1-yl) piperidine-1-carboxylate (500 mg, 1.05 equiv.) and pivalic acid (1.8 g, 10 equiv.) was added to a 2.0-5.0 mL microwave tube equipped with a stir bar. The tube was then sealed with a septum and heated to 100° C. under microwave irradiation for 1 h. LCMS analysis of the reaction mixture revealed full conversion of the starting material to the desired product (~10% boc cleavage occurred). The reaction mixture was then diluted with EtOAc and transferred to a separatory funnel containing sat. aq. NaHCO₃. The organic layer was then removed, and the aqueous layer was extracted twice with EtOAc. The combined organic layers were dried over sodium sulfate, filtered, and concentrated. The reaction mixture was then purified by reverse phase column chromatography (25% to 100% MeCN in water with. 0.1% TFA), affording the product, tert-butyl 4-(4-((5-bromoimidazo[1,2-a]pyrazin-8-yl)amino)-1H-pyrazol-1-yl) piperidine-1-carboxylate (300 mg, 0.642 mmol, 35% yield), as a tan solid. LC/MS Method 2: MS (ESI) [M] 462.3, rt: 1.66 min.

Synthesis of N-(1-(Piperidin-4-yl)-1H-pyrazol-4-yl)-5-(m-tolyl)imidazo[1,2-a]pyrazin-8-amine. A 0.5-2 mL microwave reaction tube equipped with stir bar was charged with tert-butyl 4-[4-[(5-bromoimidazo[1,2-a]pyrazin-8-yl)amino]pyrazol-1-yl]piperidine-1-carboxylate (200 mg, 0.43 mmol), m-tolyl boronic acid (120 mg, 2.0 equiv), Xphos (15.5 mg, 7.5 mol %) and XPhos-Pd-G3 (27.5 mg, 7.5 mol %). The reaction vessel was then sealed with a septum and purged with nitrogen. 1,4-Dioxane (2.2 mL, 0.2 M) and 2 M aqueous sodium carbonate (650 μL, 3 equiv.) were then added to the reaction vessel via syringe. The reaction vessel was then sealed with a septa cap and then mixed by vortex. The vessel was then heated at 135° C. under microwave irradiation for 45 min. After this time, the reaction was then allowed to cool to 22° C. and filtered through a pad of celite. The filter cake was then washed with EtOAc. LCMS analysis of the crude reaction mixture revealed full conversion of the starting material to the desired product. The reaction mixture was then purified by reverse phase column chromatography (25% to 100% MeCN in water with 0.1% TFA). The fractions were then concentrated and immediately dissolved in 1:1 DCM/TFA and allowed to stir at room temperature for 1 h, LCMS analysis revealed full deprotection to the desired product. The reaction mixture was again purified by reverse phase column chromatography (15% to 100% MeCN in water with 0.1% TFA), affording the product, N-(1-(piperidin-4-yl)-1H-pyrazol-4-yl)-5-(m-tolyl)imidazo[1,2-a]pyrazin-8-amine (141 mg, 0.379 mmol, 88% yield), as a light-yellow solid after lyophilization. MS (ESI) [M+H]⁺ 374.5. ¹H NMR (400 MHz, methanol-d₄) δ ppm 2.27-2.41 (m, 4H), 2.45 (s, 3H), 3.19-3.29 (m, 2H), 3.59 (br d, J=13.2 Hz, 2H), 4.60 (tt, J=10.1, 4.8 Hz, 1H), 7.31 (s, 1H), 7.39-7.51 (m, 4H), 7.81 (s, 2H), 7.94 (s, 1H), 8.24 (s, 1H).

### Example S11. Synthesis of 4-(Piperidin-4-yl)-N-(5-(m-tolyl)imidazo[1,2-a]pyrazin-8-yl)thiazol-2-amine (11)

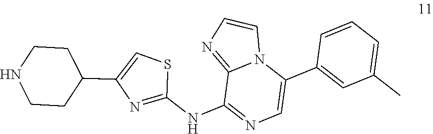 [SMILES: Cc1cccc(-c2cnc(Nc3nc(C4CCNCC4)cs3)c3nccn23)c1]

Synthesis of tert-Butyl 4-(2-((5-bromoimidazo[1,2-a]pyrazin-8-yl)amino)thiazol-4-yl) piperidine-1-carboxylate. 5,8-Dibromoimidazo[1,2-a]pyrazine (200. mg, 0.7200 mmol), tert-butyl 4-(2-aminothiazol-4-yl) piperidine-1-carboxylate (245.61 mg, 0.8700 mmol), sodium tert-butoxide (104.11 mg, 1.08 mmol), Pd₂(dba)₃ (33.07 mg, 0.0400 mmol), and Xantphos (41.79 mg, 0.070 mmol) were added to a 20 mL vial equipped with stir bar. The vial was then sealed with a septum and purged with nitrogen. Toluene (7.2 mL) was then added, and the reaction mixture was sparged with a nitrogen balloon while sonicating for 2 minutes. The reaction vessel was than heated to 100° C. and allowed to stir for 16 hours. The following morning LCMS analysis revealed formation of the desired product. The reaction mixture was than filtered, concentrated, and purified by reverse phase column chromatography. The fractions were collected and basified with sat. aq. sodium bicarbonate prior to concentration. The remaining aqueous mixture was then extracted with EtOAc. The organic layers were collected, dried over sodium sulfate, and concentrated, affording the product, tert-butyl 4-[2-[(5-bromoimidazo[1,2-a]pyrazin-8-yl)amino]thiazol-4-yl]piperidine-1-carboxylate (230 mg, 0.45 mmol, 63.1% yield) as a brown oil. LC/MS Method 2: MS (ESI) [M] 479.3, rt: 1.82 min.

Synthesis of 4-(Piperidin-4-yl)-N-(5-(m-tolyl)imidazo[1,2-a]pyrazin-8-yl)thiazol-2-amine. A 1 dram vial equipped with stir bar was charged with tert-butyl 4-[2-[(5-bromoimidazo[1,2-a]pyrazin-8-yl)amino]thiazol-4-yl]piperidine-1-carboxylate (24.0 mg, 0.050 mmol), m-tolyl boronic acid (13.6 mg, 0.10 mmol), potassium phosphate (48.94 mg, 0.1500 mmol) and Pd(dppf)Cl₂ (4.24 mg, 0.010 mmol). The reaction vessel was then sealed with a septum and purged with nitrogen. 1,4-Dioxane (0.7614 mL) and water (0.0730 mL) were then added to the reaction vessel via syringe. The reaction vessel was then sealed with a septa cap and then mixed by vortex. The vessel was then heated at 100° C. for 16 hours. After this time, the reaction was then allowed to cool to 22° C. and filtered through a pad of celite. The filter cake was then washed with EtOAc. LCMS analysis of the crude reaction mixture revealed full conversion of the starting material to the desired product. The reaction mixture was then purified by reverse phase column chromatography (25% to 100% MeCN in water with 0.1% TFA). The fractions were then concentrated and immediately dissolved in 1:1 DCM/TFA and allowed to stir at room temperature for 1 h, LCMS analysis revealed full deprotection to the desired product. The reaction mixture was again purified by reverse phase column chromatography (15% to 100% MeCN in water with 0.1% TFA), affording the product, 4-(piperidin-4-yl)-N-(5-(m-tolyl)imidazo[1,2-a]pyrazin-8-yl)thiazol-2-amine (8.9 mg, 0.022 mmol, 45% yield), as a light-yellow solid after lyophilization. MS (ESI) [(M+2H)/2]⁺ 196.5. ¹H NMR (400 MHz, methanol-d₄) δ ppm 1.90-2.03 (m, 2H), 2.30 (br d, J=12.5 Hz, 2H), 2.46 (s, 3H), 3.01-3.11 (m, 1H), 3.11-3.21 (m, 2H), 3.51 (br d, J=12.8 Hz, 2H), 6.79 (s, 1H), 7.37-7.44 (m, 1H), 7.47-7.54 (m, 3H), 7.70 (s, 1H), 7.82 (s, 1H), 7.97 (s, 1H).

### Example S12. Synthesis of N-(2-(Piperidin-4-yl)-2H-1,2,3-triazol-4-yl)-5-(m-tolyl)imidazo[1,2-a]pyrazin-8-amine (12)

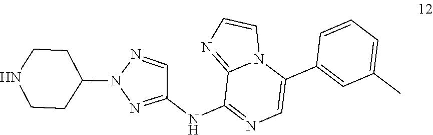 [SMILES: Cc1cccc(-c2cnc(Nc3cnn(C4CCNCC4)n3)c3nccn23)c1]

Synthesis of tert-butyl 4-(4-amino-2H-1,2,3-triazol-2-yl) piperidine-1-carboxylate. 4-Nitro-2H-triazole (489.97 mg, 4.3 mmol), t-butyl 4-(methylsulfonyloxy) piperidine-1-carboxylate (1200 mg, 4.3 mmol), and cesium carbonate (2799 mg, 8.59 mmol) were weighed out into a 40 mL vial equipped the stir bar. The reaction vessel was then sealed with a septum and the solids were suspended in DMF (7.2 mL). The reaction was then warmed to 95° C. and allowed to stir overnight. The following morning, cold water (~25 mL) was added to the reaction vessel. The precipitate was filtered and washed with cold water, and transferred to a 250 mL RBF equipped with stir bar. Then 10% palladium on carbon (200 mg, 1.88 mmol) was added and the flask was then capped with a septum followed by the addition of ethanol (18.8 mL). The vessel was then evacuated and back-filled with hydrogen gas 3 times. The reaction was allowed to stir for 4 h at room temperature. The vessel was then removed, and the flask was purged with nitrogen gas. The mixture was then filtered over a pad of celite, and the filtrate was concentrated. Purification by reverse phase silica gel chromatography (5% to 100% MeCN in water with 0.1% ammonium hydroxide) afforded the product, tert-butyl 4-(4-amino-2H-1,2,3-triazol-2-yl) piperidine-1-carboxylate (220 mg, 0.823 mmol, 19% yield), as a yellow solid. LC/MS Method 2: MS (ESI) [M+H-t-Bu]⁺ 212.5, rt: 1.29 min.

Synthesis of tert-Butyl 4-(4-((5-bromoimidazo[1,2-a]pyrazin-8-yl)amino)-2H-1,2,3-triazol-2-yl) piperidine-1-carboxylate. 5,8-dibromoimidazo[1,2-a]pyrazine (200.0 mg, 0.720 mmol), tert-butyl 4-(4-aminotriazol-2-yl) piperidine-1-carboxylate (231.7 mg, 0.870 mmol), sodium tert-butoxide (104.1 mg, 1.08 mmol), Pd₂(dba)₃ (33.1 mg, 0.040 mmol), and Xantphos (41.8 mg, 0.070 mmol) were added to a 20 mL vial equipped with stir bar. The vial was then sealed with a septum and purged with nitrogen. Toluene (7.2 mL) was then added, and the reaction mixture was sparged with a nitrogen balloon while sonicating for 2 minutes. The reaction vessel was than heated to 100° C. and allowed to stir for 16 hours. The following morning LCMS analysis revealed formation of the desired product. The reaction mixture was than filtered, concentrated, and purified by reverse phase column chromatography (5% to 100% MeCN in water with 0.1% TFA. The fractions were collected and basified with sat. aq. sodium bicarbonate prior to concentration. The remaining aqueous mixture was then extracted with EtOAc. The organic layers were collected, dried over sodium sulfate, and concentrated, affording the product, tert-butyl 4-[4-[(5-bromoimidazo[1,2-a]pyrazin-8-yl)amino]triazol-2-yl]piperidine-1-carboxylate (158 mg, 0.3069 mmol, 42.5% yield) as a brown oil. LC/MS Method 2: MS (ESI) [M]⁺ 463.4, rt: 1.75 min.

Synthesis of N-(2-(Piperidin-4-yl)-2H-1,2,3-triazol-4-yl)-5-(m-tolyl)imidazo[1,2-a]pyrazin-8-amine. A 1 dram vial equipped with stir bar was charged with tert-butyl 4-[4-[(5-bromoimidazo[1,2-a]pyrazin-8-yl)amino]triazol-2-yl]piperidine-1-carboxylate (158.0 mg, 0.340 mmol), m-tolyl boronic acid (92.7 mg, 0.680 mmol), potassium phosphate (333.3 mg, 1.02 mmol), and Pd(dppf)Cl₂ (28.9 mg, 0.030 mmol). The reaction vessel was then sealed with a septum and purged with nitrogen. 1,4-Dioxane (3.1 mL) and water (0.3 mL) were then added to the reaction vessel via syringe. The reaction vessel was then sealed with a septa cap and then mixed by vortex. The vessel was then heated at 100° C. for 16 hours. After this time, the reaction was allowed to cool to 22° C. and filtered through a pad of celite. The filter cake was then washed with EtOAc. LCMS analysis of the crude reaction mixture revealed full conversion of the starting material to the desired product. The reaction mixture was then purified by reverse phase column chromatography (25% to 100% MeCN in water with 0.1% TFA). The fractions were then concentrated and immediately dissolved in 1:1 DCM/TFA and allowed to stir at room temperature for 1 h, LCMS analysis revealed full deprotection to the desired product. The reaction mixture was again purified by reverse phase column chromatography (15% to 100% MeCN in water with 0.1% TFA), affording the product, N-(2-(piperidin-4-yl)-2H-1,2,3-triazol-4-yl)-5-(m-tolyl)imidazo[1,2-a]pyrazin-8-amine (104 mg, 0.272 mmol, 80% yield), as a white solid after lyophilization. MS (ESI) [M+H]⁺ 375.5. ¹H NMR (400 MHz, methanol-d₄) δ ppm 2.39-2.52 (m, 7H), 3.25-3.3.28 (m, 2H), 3.53-3.57 (m, 2H), 4.82-4.87 (m, 1H), 7.38-7.39 (m, 1H) 7.46-7.56 (m, 4H) 7.75 (s, 1H) 7.93 (s, 1H), 8.25 (s, 1H).

### Example S13. Synthesis of 5-(Piperidin-4-yl)-N-(5-(m-tolyl)imidazo[1,2-a]pyrazin-8-yl)thiazol-2-amine (13)

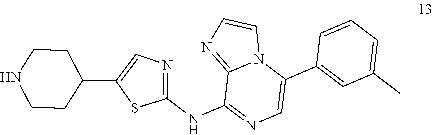 [SMILES: Cc1cccc(-c2cnc(Nc3ncc(C4CCNCC4)s3)c3nccn23)c1]

Synthesis of tert-Butyl 4-(2-((5-bromoimidazo[1,2-a]pyrazin-8-yl)amino)thiazol-5-yl) piperidine-1-carboxylate. 5,8-Dibromoimidazo[1,2-a]pyrazine (200.0 mg, 0.720 mmol), tert-butyl 4-(2-aminothiazol-5-yl) piperidine-1-carboxylate (245.6 mg, 0.87 mmol), sodium tert-butoxide (104.1 mg, 1.08 mmol), Pd₂(dba)₃ (33.07 mg, 0.040 mmol), and Xantphos (41.79 mg, 0.0700 mmol) were added to a 20 mL vial equipped with stir bar. The vial was then sealed with a septum and purged with nitrogen. Toluene (7.2 mL) was then added, and the reaction mixture was sparged with a nitrogen balloon while sonicating for 2 minutes. The reaction vessel was than heated to 100° C. and allowed to stir for 16 hours. The following morning LCMS analysis revealed formation of the desired product. The reaction mixture was than filtered, concentrated, and purified by reverse phase column chromatography. The fractions were collected and basified with sat. aq. sodium bicarbonate prior to concentration. The remaining aqueous mixture was then extracted with EtOAc. The organic layers were collected, dried over sodium sulfate, and concentrated, affording the product, tert-butyl 4-[2-[(5-bromoimidazo[1,2-a]pyrazin-8-yl)amino]thiazol-5-yl]piperidine-1-carboxylate (140 mg, 0.27 mmol, 38% yield), as a yellow powder. LC/MS Method 2: MS (ESI) [M]⁺ 479.4, rt: 1.77 min.

Synthesis of 5-(Piperidin-4-yl)-N-(5-(m-tolyl)imidazo[1,2-a]pyrazin-8-yl)thiazol-2-amine. A 1 dram vial equipped with stir bar was charged with tert-butyl 4-[2-[(5-bromoimidazo[1,2-a]pyrazin-8-yl)amino]thiazol-5-yl]piperidine-1-carboxylate (140.0 mg, 0.2900 mmol), m-tolylboronic acid (79.4 mg, 0.580 mmol), potassium phosphate (285.5 mg, 0.880 mmol), and Pd(dppf)Cl₂ (24.75 mg, 0.0300 mmol). The reaction vessel was then sealed with a septum and purged with nitrogen. 1,4-Dioxane (2.6 mL) and water (0.25 mL) were then added to the reaction vessel via syringe. The reaction vessel was then sealed with a septa cap and then mixed by vortex. The vessel was then heated at 100° C. for 16 hours. After this time, the reaction was then allowed to cool to 22° C. and filtered through a pad of celite. The filter cake was then washed with EtOAc. LCMS analysis of the crude reaction mixture revealed full conversion of the starting material to the desired product. The reaction mixture was then purified by reverse phase column chromatography (25% to 100% MeCN in water with 0.1% TFA). The fractions were then concentrated and immediately dissolved in 1:1 DCM/TFA and allowed to stir at room temp, for 1 h, LCMS analysis revealed full deprotection to the desired product. The reaction mixture was again purified by reverse phase column chromatography (15% to 100% MeCN in water with 0.1% TFA), affording the product, 5-(piperidin-4-yl)-N-(5-(m-tolyl)imidazo[1,2-a]pyrazin-8-yl)thiazol-2-amine (34.4 mg, 0.086 mmol, 29% yield), as a yellow solid, after lyophilization. MS (ESI) [M+H]⁺ 391.5. ¹H NMR (400 MHz, methanol-d₄) δ ppm 1.86-2.01 (m, 2H), 2.23-2.35 (m, 2H), 2.47 (s, 3H), 3.11-3.24 (m, 3H), 3.45-3.56 (m, 2H), 7.24 (s, 1H), 7.39-7.47 (m, 1H) 7.47-7.56 (m, 3H) 7.86 (s, 1H), 7.96 (s, 1H), 8.05 (s, 1H).

### Example S14. Synthesis of N-(1-(piperidin-4-yl)-1H-pyrazol-3-yl)-5-(m-tolyl)imidazo[1,2-a]pyrazin-8-amine (14)

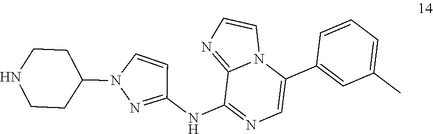 [SMILES: Cc1cccc(-c2cnc(Nc3ccn(C4CCNCC4)n3)c3nccn23)c1]

Synthesis of tert-Butyl 4-(3-((5-bromoimidazo[1,2-a]pyrazin-8-yl)amino)-1H-pyrazol-1-yl) piperidine-1-carboxylate. 5,8-Dibromoimidazo[1,2-a]pyrazine (200.0 mg, 0.720 mmol), tert-butyl 4-(3-aminopyrazol-1-yl) piperidine-1-carboxylate (230.8 mg, 0.870 mmol), sodium tert-butoxide (104.1 mg, 1.08 mmol), Pd₂(dba)₃ (33.1 mg, 0.040 mmol), and Xantphos (41.8 mg, 0.070 mmol) were added to a 20 mL vial equipped with stir bar. The vial was then sealed with a septum and purged with nitrogen. Toluene (7.2 mL) was then added, and the reaction mixture was sparged with a nitrogen balloon while sonicating for 2 minutes. The reaction vessel was than heated to 100° C. and allowed to stir for 16 hours. The following morning LCMS analysis revealed formation of the desired product. The reaction mixture was than filtered, concentrated, and purified by reverse phase column chromatography. The fractions were collected and basified with sat. aq. sodium bicarbonate prior to concentration. The remaining aqueous mixture was then extracted with EtOAc. The organic layers were collected, dried over sodium sulfate, and concentrated, affording the product, tert-butyl 4-[3-[(5-bromoimidazo[1,2-a]pyrazin-8-yl)amino]pyrazol-1-yl]piperidine-1-carboxylate (240 mg, 0.4153 mmol, 57.5% yield), as a brown powder. LC/MS Method 2: MS (ESI) [M]⁺ 462.3, rt: 1.59 min.

Synthesis of N-(1-(Piperidin-4-yl)-1H-pyrazol-3-yl)-5-(m-tolyl)imidazo[1,2-a]pyrazin-8-amine. A 1 dram vial equipped with stir bar was charged with tert-butyl 4-[3-[(5-bromoimidazo[1,2-a]pyrazin-8-yl)amino]pyrazol-1-yl]piperidine-1-carboxylate (240.0 mg, 0.520 mmol), m-tolylboronic acid (141.2 mg, 1.04 mmol), potassium phosphate (507.4 mg, 1.56 mmol), and Pd(dppf)Cl₂ (44.0 mg, 0.050 mmol). The reaction vessel was then sealed with a septum and purged with nitrogen. 1,4-Dioxane (4.7 mL) and water (0.46 mL) were then added to the reaction vessel via syringe. The reaction vessel was then sealed with a septa cap and mixed by vortex. The vessel was then heated at 100° C. for 16 hours. After this time, the reaction was then allowed to cool to 22° C. and filtered through a pad of celite. The filter cake was then washed with EtOAc. LCMS analysis of the crude reaction mixture revealed full conversion of the starting material to the desired product. The reaction mixture was then purified by reverse phase column chromatography (25% to 100% MeCN in water with 0.1% TFA). The fractions were then concentrated and immediately dissolved in 1:1 DCM/TFA and allowed to stir at room temperature for 1 h, LCMS analysis revealed full deprotection to the desired product). The reaction mixture was again purified by reverse phase column chromatography (15% to 100% MeCN in water with 0.1% TFA), affording the product, N-(1-(piperidin-4-yl)-1H-pyrazol-3-yl)-5-(m-tolyl)imidazo[1,2-a]pyrazin-8-amine (162 mg, 0.41 mmol, 79% yield), as a light-yellow solid after lyophilization. LC/MS Method 2: MS (ESI) [M+H]⁺ 374.5, rt: 1.11 min. ¹H NMR (400 MHz, methanol-d₄) δ ppm 2.25-2.42 (m, 4H), 2.45 (s, 3H), 3.08-3.22 (m, 2H), 3.51-3.61 (m, 2H), 4.62-4.73 (m, 1H), 6.39-6.48 (m, 1H), 7.33 (s, 1H), 7.35-7.41 (m, 1H), 7.41-7.51 (m, 3H), 7.60-7.66 (m, 1H), 7.73-7.80 (m, 1H), 7.90-7.96 (m, 1H).

### Example S15. Synthesis of 3-(Piperidin-4-yl)-N-(5-(m-tolyl)imidazo[1,2-a]pyrazin-8-yl)isoxazol-5-amine (15)

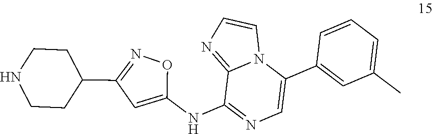 [SMILES: Cc1cccc(-c2cnc(Nc3cc(C4CCNCC4)no3)c3nccn23)c1]

Synthesis of tert-Butyl 4-(5-((5-bromoimidazo[1,2-a]pyrazin-8-yl)amino)isoxazol-3-yl) piperidine-1-carboxylate. 5,8-Dibromoimidazo[1,2-a]pyrazine (200.0 mg, 0.720 mmol), tert-butyl 4-(5-aminoisoxazol-3-yl) piperidine-1-carboxylate (231.7 mg, 0.870 mmol), sodium tert-butoxide (104.1 mg, 1.08 mmol), Pd₂(dba)₃ (33.1 mg, 0.040 mmol), and Xantphos (41.8 mg, 0.070 mmol) were added to a 20 mL vial equipped with stir bar. The vial was then sealed with a septum and purged with nitrogen. Toluene (7.2 mL) was then added, and the reaction mixture was sparged with a nitrogen balloon while sonicating for 2 minutes. The reaction vessel was than heated to 100° C. and allowed to stir for 16 hours. The following morning LCMS analysis revealed formation of the desired product. The reaction mixture was than filtered, concentrated, and purified by reverse phase column chromatography (5% to 100% MeCN in water with 0.1% TFA). The fractions were collected and basified with sat. aq. sodium bicarbonate prior to concentration. The remaining aqueous mixture was then extracted with EtOAc. The organic layers were collected, dried over sodium sulfate, and concentrated, affording the product, tert-butyl 4-[5-[(5-bromoimidazo[1,2-a]pyrazin-8-yl)amino]isoxazol-3-yl]piperidine-1-carboxylate (145 mg, 0.3067 mmol, 42.465% yield), as an off-white solid. LC/MS Method 2: MS (ESI) [M+Na]⁺ 485.3, rt: 1.75 min.

Synthesis of 3-(Piperidin-4-yl)-N-(5-(m-tolyl)imidazo[1,2-a]pyrazin-8-yl)isoxazol-5-amine. A 1 dram vial equipped with stir bar was charged with tert-butyl 4-[5-[(5-bromoimidazo[1,2-a]pyrazin-8-yl)amino]isoxazol-3-yl]piperidine-1-carboxylate (145.0 mg, 0.310 mmol), m-tolylboronic acid (85.1 mg, 0.630 mmol), potassium phosphate (306 mg, 0.940 mmol), and Pd(dppf)Cl₂ (26.5 mg, 0.030 mmol). The reaction vessel was then sealed with a septum and purged with nitrogen. 1,4-Dioxane (2.8 mL) and water (0.28 mL) were then added to the reaction vessel via syringe. The reaction vessel was then sealed with a septa cap and then mixed by vortex. The vessel was then heated at 100° C. for 16 hours. After this time, the reaction was then allowed to cool to 22° C. and filtered through a pad of celite. The filter cake was then washed with EtOAc. LCMS analysis of the crude reaction mixture revealed full conversion of the starting material to the desired product. The reaction mixture was then purified by reverse phase column chromatography (25% to 100% MeCN in water with 0.1% TFA). The fractions were then concentrated and immediately dissolved in 1:1 DCM/TFA and allowed to stir at room temp, for 1 h, LCMS analysis revealed full deprotection to the desired product. The reaction mixture was again purified by reverse phase column chromatography (15% to 100% MeCN in water with 0.1% TFA), affording the product, 3-(piperidin-4-yl)-N-(5-(m-tolyl)imidazo[1,2-a]pyrazin-8-yl)isoxazol-5-amine (23.6 mg, 0.061 mmol, 19% yield), as a tan solid, after lyophilization. LC/MS Method 2: MS (ESI) [M+H]⁺ 375.5, rt: 1.14 min. 1H NMR (400 MHz, methanol-d₄) δ ppm 2.01-2.14 (m, 2H), 2.26-2.36 (m, 2H), 2.48 (s, 3H), 3.10-3.21 (m, 3H), 3.44-3.53 (m, 2H), 6.65 (s, 1H), 7.36-7.41 (m, 1H), 7.42-7.52 (m, 3H), 7.52-7.56 (m, 1H), 7.66-7.72 (s, 1H), 7.82-7.88 (s, 1H).

### BIOLOGICAL EXAMPLES

### Example B1. IRAK3 Biochemical Binding Assay

The LanthaScreen® Eu Kinase Binding assay was performed as described by the vendor (ThermoFisher Scientific Waltham, MA). Briefly, 100× solutions of compound were prepared in DMSO via serial dilution of the 10 mM stock solution in a 384-well reagent plate using 3-fold intervals to achieve final concentrations. 1 μL of the compound dilution series were added to the corresponding wells of a 384-well reagent plate containing 32.3 μL of 1× buffer (50 mM HEPES pH 7.4, 10 nM MgCl₂, 1 mM EGTA, 0.01% Brij-35). 5 μL of the buffer diluted compounds were transferred to the corresponding wells of a 384-well assay plate. 5 μL of 3× tracer was transferred to each well of the assay plate for a final tracer concentration of 10 nM. Finally, 5 μL of the 3×Eu-Anti-GST and IRAK3 mix was transferred to each well for a final concentration of 2 nM and 10 nM respectively. Reactions were allowed to incubate for 1 hour at room temperature. TR-FRET signal of the interaction (λex340/λem665/λem615) was read at room temperature with a delay time of 100 μs and an integration time of 200 μs using an Envision plate reader. Using this assay, IC₅₀ values of the following compounds were determined. Results are shown in Table 2.

TABLE 2
IRAK3 biochemical binding assay.

| Compound No. | IRAK3 TR-FRET IC₅₀ (nM) |
|---|---|
| 1 | 4680 |
| 2 | 60 |
| 3 | 109 |
| 4 | 393 |
| 5 | 22 |
| 6 | 448 |
| 7 | 88 |
| 8 | 32 |
| 9 | 24 |
| 10 | 5 |
| 11 | 2499 |
| 12 | 59 |
| 13 | 1093 |
| 14 | >10000 |
| 15 | 100 |

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A compound of Formula (I): 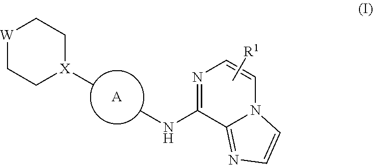
or a pharmaceutically acceptable salt thereof, wherein:
Ring A is C₆-C₁₀ aryl or 5- to 6-membered heteroaryl, wherein the heteroaryl contains 1-3 heteroatoms selected from nitrogen, oxygen, and sulfur;
R¹ is H, C₆-C₁₀ aryl, or C₃-C₆ cycloalkyl, wherein the aryl and cycloalkyl are optionally substituted by 1-5 substituents selected from C₁-C₆ alkyl, C₁-C₆ haloalkyl, C₁-C₆ alkoxy, halo, —OH, and —CN;
W is NR² or CR^{3a}R^{3b},
R² is H, C₁-C₆ alkyl, C₁-C₆ haloalkyl, or —C(O)(C₁-C₆ alkyl);
R^{3a} and R^{3b} are independently H, C₁-C₆ alkyl, C₁-C₆ haloalkyl, C₁-C₆ alkoxy, or —C(O)(C₁-C₆ alkyl); and
X is CH or N.

2. The compound of claim 1, or a pharmaceutically acceptable salt thereof, wherein:
Ring A is C₆-C₁₀ aryl.

3. The compound of claim 1, or a pharmaceutically acceptable salt thereof, wherein:
Ring A is 5- to 6-membered heteroaryl, wherein the heteroaryl contains 1-3 heteroatoms selected from nitrogen, oxygen, and sulfur.

4. The compound of any one of claims 1-3, or a pharmaceutically acceptable salt thereof, wherein:
Ring A is

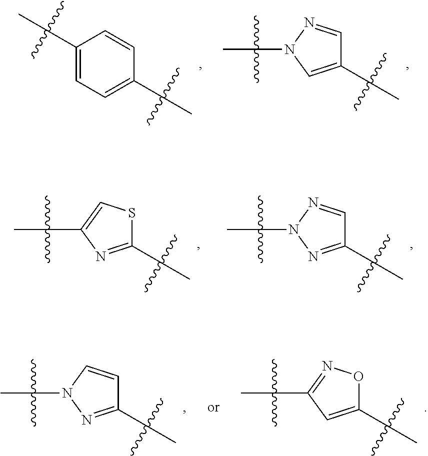 [SMILES: *c1ccc(*)cc1 ; *c1cnn(*)c1 ; *c1csc(*)n1 ; *c1cnn(*)n1 ; *c1ccn(*)n1 ; *c1cc(*)on1]

5. The compound of any one of claims 1-4, or a pharmaceutically acceptable salt thereof, wherein R¹ is H.

6. The compound of any one of claims 1-4, or a pharmaceutically acceptable salt thereof, wherein R¹ is C₃-C₆ cycloalkyl optionally substituted by 1-5 substituents selected from C₁-C₆ alkyl, C₁-C₆ haloalkyl, C₁-C₆ alkoxy, halo, —OH, and —CN.

7. The compound of claim 6, or a pharmaceutically acceptable salt thereof, wherein R¹ is C₃-C₆ cycloalkyl.

8. The compound of any one of claims 1-4, or a pharmaceutically acceptable salt thereof, wherein R¹ is C₆-C₁₀ aryl optionally substituted by 1-5 substituents selected from C₁-C₆ alkyl, C₁-C₆ haloalkyl, C₁-C₆ alkoxy, halo, —OH, and —CN.

9. The compound of any one of claims 1-4, 6, and 7, or a pharmaceutically acceptable salt thereof, wherein:
R¹ is

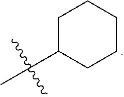 [SMILES: *C1CCCCC1]

10. The compound of any one of claims 1-4 and 8, or a pharmaceutically acceptable salt thereof, wherein:
R¹ is

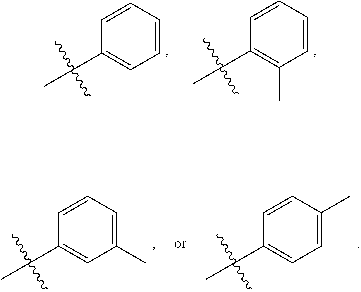

11. The compound of any one of claims 1-10, or a pharmaceutically acceptable salt thereof, wherein:
W is NR² or CR^{3a}R^{3b};
R² is H, C₁-C₆ alkyl, or —C(O)(C₁-C₃ alkyl); and
R^{3a} and R^{3b} are independently H, C₁-C₃ alkyl, or —C(O)(C₁-C₆ alkyl).

12. The compound of claim 11, or a pharmaceutically acceptable salt thereof, wherein:
W is NR²; and
R² is H, C₁-C₆ alkyl, or —C(O)(C₁-C₃ alkyl).

13. The compound of any one of claims 1-10, or a pharmaceutically acceptable salt thereof, wherein X is CH.

14. The compound of any one of claims 1-10, or a pharmaceutically acceptable salt thereof, wherein X is N.

15. The compound of any one of claims 1-13, or a pharmaceutically acceptable salt thereof, wherein: 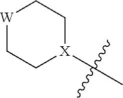
is

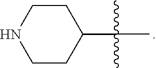 [SMILES: *C1CCNCC1]

16. The compound of any one of claims 1-12 and 14, or a pharmaceutically acceptable salt thereof, wherein: 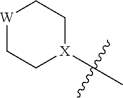
is

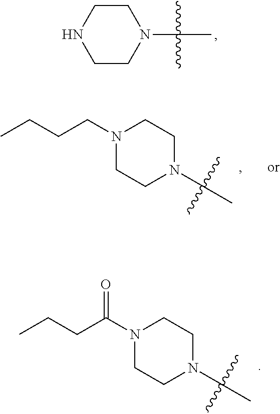 [SMILES: *N1CCNCC1 ; *N1CCN(CCCC)CC1 ; *N1CCN(C(=O)CCC)CC1]

17. The compound of any one of claims 1-16, or a pharmaceutically acceptable salt thereof, wherein the compound is Formula (II) or (III): 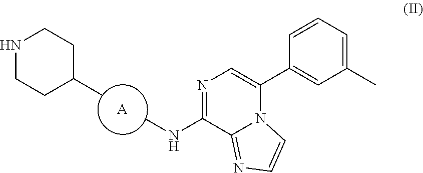
wherein Ring A is 5-membered heteroaryl that contains 1-3 heteroatoms selected from nitrogen, oxygen, sulfur;

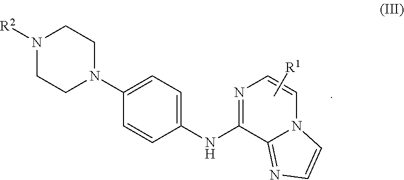

18. A compound selected from the compounds of Table 1 or a pharmaceutically acceptable salt thereof.

19. A pharmaceutical composition comprising the compound of any one of claims 1-18, or a pharmaceutically acceptable salt thereof, and a pharmaceutically acceptable excipient.

20. A method of binding Interleukin-1 Receptor-Associated Kinase 3 (IRAK3) comprising contacting IRAK3 with an effective amount of the compound of any one of claims 1-18, or a pharmaceutically acceptable salt thereof, or the pharmaceutical composition of claim 19.
